(19) United States
(12) Patent Application Publication
(10) Pub. No.: US 2020/0016125 A1
(43) Pub. Date: Jan. 16, 2020

(54) GRANZYME B INHIBITOR COMPOSITIONS AND METHODS FOR THE PREVENTION AND/OR TREATMENT OF SKIN BLISTERING AND/OR PEELING

(71) Applicant: The University of British Columbia, Vancouver (CA)

(72) Inventors: David James Granville, Port Coquitlam (CA); Valerio Russo, Vancouver (CA); Yue Shen, Burnaby (CA)

(73) Assignee: The University of British Columbia, Vancouver, BC (CA)

(21) Appl. No.: 16/489,117

(22) PCT Filed: Feb. 28, 2018
(86) PCT No.: PCT/CA2018/050230

§ 371 Date: Aug. 27, 2019

Related U.S. Application Data
(60) Provisional application No. 62/611,188, filed on Dec. 28, 2017.
(60) Provisional application No. 62/465,055, filed on Feb. 28, 2017.

(51) Int. Cl. A61K 31/4166; C07K 5/062; C07K 5/083; A61P 17/00

(52) U.S. Cl. CPC A61K 31/4166; C07K 5/06026; A61K 45/06; A61P 17/00; C07K 5/0808

(57) ABSTRACT

Methods for using compositions comprising a Granzyme B inhibitor and a pharmaceutically acceptable carrier for treating and/or preventing blistering and/or peeling of a skin of a subject are provided. Also provided are methods for using the compositions to improve the healing of a blistered or area of peeled skin of a subject. The compositions can be formulated for oral administration, nasal administration, topical administration, subcorneal administration, intra-epidermal administration, sub-epidermal administration; or for administration by injection.

### BACKGROUND OF THE INVENTION

The skin consists of two main layers: the epidermis and the epidermis. Blisters are accumulations of fluid within or under the dermis. Peeling refers to damage and loss of the upper layer (epidermis) of skin. Skin peeling and/or blistering can be caused by environmental factors that irritate or damage skin such as extreme temperature (hot or cold), infection, friction, sun, wind, heat, dryness and excessive humidity, repetitive irritation, chemicals, allergens, or drugs; as well as pathological conditions including autoimmune and genetic disorders.

Blistering is a hallmark of many dermatological conditions, and can manifest itself with varying degrees of severity, but is typically characterized by erosions or fluid filled elevations from the skin surface caused by disruption of the cell to cell attachment in different layers of the epidermis, or detachment of the epidermis from dermis. Due to the critical role that skin plays as a barrier in regulating fluid/electrolyte retention, thermoregulation, and protection against infection, depending on the size and severity of blistering, such functions can be compromised and potentially fatal (Wong et al., Australas J. Dermatol. 40:131-134, 1999).

Based on the etiology, these dermatoses are generally classified in four major groups: a) antibody-mediated, b) cutaneous adverse drug reactions, c) congenital conditions, and d) blistering caused by external insults such as burns, friction, sunlight, insect bites, infections, and chemical weapons. With respect to autoimmune skin blistering diseases, auto-antibodies are produced against structural or adhesive molecules of the skin and based on the location of the specific auto-antigens and level of blister formation; these diseases are further classified into intra-epidermal and sub-epidermal blistering diseases. Pemphigus (intra-epidermal blistering) is characterized by interepithelial blistering that is characterized by a loss of cell-cell adhesion (acantholysis) and the presence of antibodies specific against epithelial adhesion proteins such as desmogleins, cadherins and/or other desmosomal proteins. In sub-epidermal blistering dermatoses such as bullous pemphigoid, dermatitis herpetiformis and epidermolysis bullosa, auto-antibodies targeting components of the dermal-epidermal junction (DEJ) lead to the disruption of this basement membrane and consequent detachment of the epidermis (Baum et al., Autoimmun. Rev. 13:482-489, 2014).

Peeling skin syndrome (also referred to as deciduous skin, familial continuous skin peeling, exfoliative ichthyosis) refers to a group of rare inherited skin disorders characterized by painless, continual, spontaneous skin peeling (exfoliation) due to a separation of the stratum corneum (outermost layer of skin) from the underlying epidermis. Peeling skin syndromes can also exhibit blistering and/or erythema (skin reddening) and/or pruritus (itching). Symptoms of peeling skin syndromes can be observed at birth or can appear in early childhood. Two forms of peeling skin syndrome are recognized: (1) a generalized form involving the entire integument; and (2) an acral form (acral peeling skin syndrome) involving only the extremities, and mostly hands and feet. In the acral forms, patients usually develop blisters and erosions on hands and feet at birth or during infancy, which is reminiscent of the blistering skin disorder, epidermolysis bullosa simplex.

Blistering skin disorders are a group of rare skin diseases involving blistering and erosions in the skin and/or mucous membranes. Types of blistering skin diseases include: (1) autoimmune blistering diseases such as bullous pemphigoid, drug-induced pemphigus, endemic pemphigus, pemphigus erythematosus, pemphigus vegetans, pemphigus vulgaris, pemphigus foliaceus, paraneoplastic pemphigus, mucous membrane pemphigoid, epidermolysis bullosa, Linear IgA disease, bullous lupus, dermatitis herpetiformis and (2) genetic blistering diseases such as epidermolysis bullosa (EB) including for example, epidermolysis bullosa simplex, junctional epidermolysis bullosa, dystrophic epidermolysis bullosa, and epidermalysis bullosa acquisita. Sometimes, the skin will blister when it comes into contact with a cosmetic, detergent, solvent, or other chemical such as Balsam of Peru, nickel sulfate, or urushiol (poison oak, poison sumac, poison ivy). Blisters also occur due to allergic reactions caused by insect bites, extracts or stings. Chemical warfare agents (i.e., blister agents) or vesicants, can also cause large, painful blisters wherever they contact skin (e.g., mustard gas). Blistering can also occur after contact with several types of beetles that release vesicants such as Cantharidin. Such blistering is associated with blistering beetle dermatitis or paederus dermatitis.

The dermal-epidermal junction (DEJ) is a specialized basement membrane between the epidermis and the dermis, which serves critical purposes for the integrity and function of the skin. It provides firm anchorage between the basal layer of the epidermis and the papillary layer of the dermis, while acting as a selective filter during cellular and molecular exchange between these two layers. Moreover, DEJ interaction with the basal layer of the epidermis determines the polarity of basal keratinocytes, maintaining proliferating cells attached to it while allowing daughter cells to migrate toward upper layers.

Structurally, the DEJ can be divided into 4 zones: 1) the basal epidermal cell membrane 2) the lamina lucida, 3) the lamina densa and 4) the fibrillar zone (papillary dermis). The first layer is represented by basal keratinocytes membrane and their specialized junctional structures called hemidesmosome, which connect keratinocytes cytoskeleton to the lamina lucida through the trans-membrane proteins α6/β4 and α3/β1 integrins, as well as collagen XVII. In the lamina lucida these proteins bridge the keratin cytoskeleton to the anchoring filaments mainly composed of laminin-5. These anchoring filaments, in turn, bind to collagen IV, the main component of the lamina densa, through nidogen and perlecan. The last junction in the DEJ is represented by the bond between elements in the lamina densa and the anchoring fibrils in the fibrillar zone. Burgeson and Christiano, Curr. Opin. Biol. 9:651-658, 1997; Aumailley and Rousselle Matrix Biol. 18(1):19-28, 1999.

Anchoring fibrils are almost exclusively composed of Collagen VII, a non-fibrillar collagen instrumental for the structural integrity of the DEJ. Encoded by the gene COL7A1, collagen VII is a homotrimer of three al chains and consists of one central collagenous domain flanked by a C-terminal non-collagenous domain 2 (NC-2) and a N-terminal non-collagenous domain 1 (NC-1). The latter contains several subdomains with high homologies to adhesion proteins: one cartilage matrix protein like domain (CMP), nine fibronectin-like domains (FNIII), and one von-Willebrand-factor-A like domain (vWFA2) (Leineweber et al., Febs Lett. 585:1748-1752, 2011). Among these domains, FNIII regions are pivotal for the interaction with several ECM components. Studies with a recombinant version of the NC1 region revealed strong binding affinity of FNIII domains with collagen I, collagen IV, laminin-5 and fibronectin. Chen et al., J. Biol. Chem. 272:14516-14522, 1997; Chen et al., J. Invest. Dermatol. 112:177-183, 1999. It is this region that allows anchorage of the papillary dermis to the lamina densa through FNIII binding to laminin-5 and collagen IV.

Due to its role as the first point of attachment between the dermis and the epidermis, any structural modification or functional impairment of collagen VII results in disruption of the DEJ with consequent epidermal detachment and blistering. Mutations of collagen VII or production of auto-antibodies against collagen VII, are the main cause of dystrophic epidermolysis bullosa and epidermolysis bullosa acquisita respectively. These conditions are characterized by extensive epidermal detachment and formation of blisters. Has, Curr. Top. Membr. 76:117-170, 2015.

Granzyme B (GzmB)-mediated cleavage of collagen VII after residue D390 in the FNIII-2 domain is shown herein. In addition, inhibition of this cleavage by the specific GzmB inhibitors Compound A and Compound 20 (Willoughby et al., Bioorg. Med. Chem. Lett., 12:2197-2200, 2002) is also demonstrated herein. These observations implicate GzmB in the disruption of the fibrillar zone/lamina densa connection and thus in epidermal detachment. Such GzmB-mediated DEJ disruption can occur not only during auto-immune/genetic disorders but also during blistering inflammatory events of the skin as a consequence of burns, bug bites and radiation.

In addition, α6/β4 integrin, a major collagen and laminin binding protein is identified herein as a substrate for GzmB. Evidence for the importance of this integrin comes from both animal models and from severe, often lethal, human blistering diseases. Homozygous β4 null mice die shortly after birth and display extensive detachment of the epidermis. This β4 deficiency-induced DEJ disruption is a consequence of impaired formation of hemidesmosomes at the basal surface of keratinocytes, suggesting that this integrin does not play strictly adhesive roles, but is also crucial for the assembly of the hemidesmosomes. (van der Neut et al., Nat. Genet. 13:366-369, 1996).

In humans, mutations and deficiencies of the integrin complex involving α6/β4, as well as production of auto-antibodies against α6/β4, can cause potentially lethal phenotypes characterized by widespread mucocutaneous blistering and extracutaneous involvement. Several studies have identified mutations in the genes coding for α6/β4 integrin (Pulkkinen et al., Hum. Mol. Genet. 6:669-674, 1997; Ruzzi et al., J. Clin. Invest. 99:2826-2831, 1997; Vidal et al., Nat. Genet. 10:229-234, 1995) or absence at the protein level of one of the sub-units (Niessen et al., J. Cell Sci. 109:1695-1706, 1996), in patients affected by junctional epidermolysis bullosa with pyloric atresia (JEB-PA), junctional epidermolysis bullosa gravis, and non-lethal junctional epidermolysis bullosa. Phillips et al. reported that while one antibody directed against a specific epitope of β4 showed immunoreactivity at the DEJ level in skin sections from these patients, another antibody directed against a different epitope did not. (Histopathology 24:571-576, 1994). They speculated that in situ proteolytic cleavage by an as yet unidentified protease, of the epitopes might be responsible for the loss of immunoreactivity.

Pemphigoid is a family of autoimmune disorders characterized by skin rashes and blistering on legs, arms and abdomen. Auto-antibodies against both α6 and β4 sub-units have been detected in patients with oral pemphigoid and cicatricial pemphigoid. (Sami et al., Clin. Exp. Immunol. 129:533-540, 2002; Leverkus et al., Br. J. Dermatol. 145:998-1004, 2001). Mutations of α6/β4 integrin and collagen VII are associated with blistering skin diseases. Such mutations can alter the structure of these proteins thereby predisposing the DEJ to GzmB cleavage and/or this proteolytic activity might generate autoantigens capable of causing or exacerbating auto-immune conditions.

Despite the advances in development of blistering and peeling treatments, a need exists for new treatments and improved formulations for use in preventing and/or treating skin peeling and/or blistering. The present invention seeks to fulfill these needs and provide further related advantages.

### SUMMARY OF THE INVENTION

The present invention provides compositions and methods for the treatment of blistering and skin peeling. The compositions comprise a GzmB inhibitor and a pharmaceutically acceptable carrier.

In one aspect, the invention provides compositions that include a GzmB inhibitor compound effective for preventing skin peeling and/or blistering. In one embodiment, the invention provides a formulation for skin peeling and/or blistering, comprising 4-(((2S,3S)-1-((2-((S)-5-(((2H-tetrazol-5-yl)methyl)carbamoyl)-3-cyclohexyl-2-oxoimidazolidin-1-yl)-2-oxoethyl)amino)-3-methyl-1-oxopentan-2-yl)amino)-4-oxobutanoic acid (an embodiment of Compound A) or a pharmaceutically acceptable salt thereof in combination with a pharmaceutically acceptable carrier.

In another aspect of the invention, methods for treating skin peeling and/or blistering are provided. In the methods, a composition comprising a GzmB inhibitor compound or a pharmaceutically acceptable salt thereof in combination with a pharmaceutically acceptable carrier is administered to a subject in need thereof.

In a further aspect, the invention provides methods for preventing skin peeling and/or blistering. In the methods, a composition comprising a GzmB inhibitor compound or a pharmaceutically acceptable salt thereof in combination with a pharmaceutically acceptable carrier is administered to the damaged skin.

In a further aspect of the invention, the invention provides methods for subcutaneous or intradermal delivery of a GzmB inhibitor. In the methods, a composition comprising a GzmB inhibitor compound or a pharmaceutically acceptable salt thereof in combination with a pharmaceutically acceptable carrier is administered to the skin.

In the above methods, the composition can be formulated as a gel or solution containing the GzmB inhibitor or a pharmaceutically acceptable salt thereof in combination with a pharmaceutically acceptable carrier. The gels can be orally or topically administered, and the solutions can be administered topically or by injection.

In still other embodiments the GamB inhibitor or a pharmaceutically acceptable salt thereof in combination with a pharmaceutically acceptable carrier can be administered intravenously, intramuscularly, intranasally, orally, intrathecally, or mucosally, and the like. For example, the composition can be in the form of a tablet, capsule, gel or solution.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIG. 1 shows the addition of GzmB (200 nM) to fresh human skin and separation of the DEJ. Normal skin and vehicle-treated skin is shown in first two panels. Arrow indicates separation of the epidermis from the dermis in the GzmB-treated skin. Abdominal full thickness skin was obtained from elective plastic surgery and used immediately after excision. A small piece (0.2 by 0.4 mm) was immediately fixed in 10% formalin (native skin) while other pieces were placed in 300 μL of phosphate buffer saline (PBS) with or without 200 nM GzmB. Samples were then incubated in a water bath at 37° C. for 24 hours. Following incubation the skin was fixed in 10% formaline, paraffin embedded, sectioned (5 μm) and stained with Hematoxylin and Eosin (H&E) using standard methods.

FIG. 2 shows the addition of GzmB (100 nM) to fresh human skin and separation of the DEJ. Normal skin and vehicle-treated skin are shown in first two panels. Arrow indicates separation of the epidermis from the dermis. Abdominal full thickness skin was obtained from elective plastic surgery and used immediately. Small pieces (0.2 by 0.4 mm) were placed in 300 μL of PBS with or without GzmB. Samples were then incubated in a water bath at 37° C. for 24 hours. Following incubation, the skin was fixed in 10% formalin, paraffin embedded, sectioned (5 μm), and stained with H&E using standard methods.

FIG. 3 demonstrates GzmB cleaves collagen VII. Addition of GzmB inhibitors (Serpin A3N, Compound 20, Compound A) prevented GzmB-mediated collagen VII cleavage. Bands indicating fragments are labeled. GzmB cleavage assay was performed in 40 μL of PBS. Briefly, collagen VII (500 ng) was incubated with 200 mM of GzmB for 24 hours at 37° C. For inhibition, GzmB was incubated with Compound 20, Serpin A3N and Compound A for 1 hour at 37° C. prior to the addition of collagen VII. After 24 hours, all samples were loaded onto a 10% SDS-PAGE, blotted on a PVDF membrane and incubated with a primary antibody specific for collagen VII (rabbit anti-collagen VII, Abcam plc) overnight at 4° C., and then with a secondary labeled anti-rabbit antibody for 1 hour at room temperature.

FIG. 4 is a graphic illustration of collagen VII structure. GzmB cleaves after residue D390 in the FNIII domain 2. FN-like domains are responsible for collagen VII interaction with the extracellular matrix. Cleavage sites within collagen VII for GzmB were determined using TAILS analysis using methods previously described by Kleifeld et al. (Nat. Biotechnol. 28:281-288. doi: 10.1038/nbt.1611, 2010). Briefly, collagen VII was incubated with 200 nM of GzmB for 24 hours at 37° C. N-termini were differentially labeled, denatured, and blocked. Samples were then incubated with trypsin to generate tryptic peptides and then the amine-reactive polymer HPG-ALD to negatively enrich labeled peptides and identify GzmB-cleavage sites.

FIG. 5 illustrates immunostaining of collagen VII. Arrow indicates collagen VII staining. GzmB skin cleavage assay and paraffin embedding were performed as described for FIGS. 1 and 2. Subsequently, 5 μm sections were deparaffinized and subjected to enzymatic antigen retrieval with trypsin for 15 min using the Carenzyme I: Trypsin Kit (BioCare Medical). Slides were then blocked with 10% goat serum in Tris Buffered Saline (TBS) for 1 hour prior to incubation with rabbit anti-collagen VII antibody (Abcam plc) overnight at 4° C. All slides were then incubated with secondary biotinylated antibody and DAB staining was performed following the manufacturer's instructions.

FIG. 6 shows GzmB cleavage of α6β4. GzmB inhibitors (Compound 20, Serpin A3N, and Compound A) were used. A GzmB cleavage assay was performed in L of PBS. Briefly, α6β4 (500 ng) was incubated with 200 nM of GzmB for 24 hours at 37° C. For inhibition, GzmB was incubated with Compound 20, Serpin A3N and Compound A for 1 hour at 37° C. prior to the addition of α6β4 integrin. After 24 hours, all samples were loaded onto a 10% polyacrylamide gel and separated by electrophoresis for 1.5 hours. Bands were then visualized by staining with a coomassie stain.

FIG. 7 demonstrates GzmB cleaves cell-cell adhesion proteins including the TJ protein, JAM-A, the AJ protein, E-cadherin, and the desmosomes, Dsg-1 and Dsg-3. Each protein was incubated with GzmB for 2 hours prior to being separated via SDS-PAGE to identify cleavage fragments. All five proteins showed a reduction of whole protein and an increase in fragmentation when incubated with GzmB versus protein alone or GzmB+Compound 20 inhibitor. E-cadherin and Dsg-1 showed an almost complete loss of whole protein with 100 nM GzmB, whereas JAM-A, ZO-1, and Dsg-3 showed less cleavage as whole protein was still detectable after GzmB treatment.

FIGS. 8A through 8C provide evidence Compound A, a GzmB inhibitor, prevents blistering. Mice were exposed to burn injury by heating a steal rod for 6 seconds to 100° C. to induce blistering. Two independent experiments were run: In FIG. 8A, Compound A was added daily for 30 days. The figure shows separation of the epidermis from the dermis forming a blister in saline-treated skin. Blistering is prevented in Compound A-treated skin. In FIG. 8B, Compound A in PBS (3.6 mg/mL) was administered daily by subcutaneous injection and in a gel (3.6 mg/mL) administered daily by topical application. Compound A in both formulations was found to reduce blistering compared to saline-only treatment (administered daily by subcutaneous injection). In FIG. 8C, Compound A in gel (3.6 mg/mL) administered daily through topical application reduced blistering as compared to gel alone (administered daily by topical application).

FIG. 9 is a schematic illustration of a representative synthetic pathway for the preparation of representative compounds (P5-P4-P3-P2-P1 starting from P1) useful in the formulations and methods of the invention.

FIG. 10 is a schematic illustration of another representative synthetic pathway for the preparation of representative compounds (P5-P4-P3-P2-P1 starting from P5) useful in the formulations and methods of the invention.

FIG. 11 is a schematic illustration of a further representative synthetic pathway for the preparation of representative compounds (P5-P4-P3-P2-P1 starting from a component other than P1 or P5) useful in the formulations and methods of the invention.

FIGS. 12A and 12B demonstrate GzmB levels are elevated in the DEJ of sub-epidermal blistering diseases. FIG. 12A depicts in the upper row, representative images of H&E staining of healthy skin, bullous pemphigoid (BP), dermatitis herpetiformis (DH), and Epidermolysis Bullosa Acquisita (EBA). In the lower row, GzmB immunostaining of healthy, bullous pemphigoid (BP), dermatitis herpetiformis (DH), and Epidermolysis Bullosa Acquisita (EBA) biopsies are shown. Abundant GzmB is observed at the level of the dermal-epidermal junction in diseased skin particularly in areas of epidermal separation (grey arrowheads). Dotted lines indicate separation between the epidermis and the dermis. Scale bars represent 200 μm. FIG. 12B depicts in the upper row, representative images of H&E staining of, bullous pemphigoid (BP), dermatitis herpetiformis (DH), and Epidermolysis Bullosa Acquisita (EBA). In the lower row, GzmB staining for the same tissue sections is provided. In all conditions studied GzmB co-localizes with neutrophils (gray circles). Scale bars represent 40 μm.

FIGS. 13A through 13C demonstrate that α6 integrin is a GzmB substrate and is reduced in sub-epidermal blistering. FIG. 13A depicts 4-20% SDS-PAGE Western Blot analysis of GzmB-mediated cleavage of the α6 integrin (α6 int) subunit with and without inhibitors serpin A3N (SA3N) and Compound 20 (Com20). Black arrows indicate cleavage fragments and * indicates full length proteins. At a concentration of 200 nM, GzmB produces cleavage bands, and this cleavage is prevented by GzmB inhibitors. FIG. 13B depicts an extracellular domain schematic for α6 integrin. GzmB mediated cleavage sites were identified proteomically by ATOMs (grey arrows) fall within the ligand-binding domains in the n-propeller region. GzmB, granzyme B; TM, transmembrane helix. FIG. 13C demonstrates α6 integrin immunostaining of healthy, bullous pemphigoid (BP), dermatitis herpetiformis (DH), and Epidermolysis Bullosa Acquisita (EBA) biopsies. Grey arrowheads indicate intact α6 integrin in areas of dermo-epidermal adhesion, black arrowheads indicate weak or absent staining. Dotted lines indicate separation between the epidermis and the dermis. Scale bars represent 200 μm.

FIGS. 14A through 14C demonstrates β4 integrin cleavage by GzmB and status in healthy skin versus and sub-epidermal blistering. FIG. 14A depicts a 4-20% SDS-PAGE Western Blot of GzmB-mediated cleavage of β4 integrin sub-unit (β4 int) with and without inhibitors serpin A3N (SA3N) and Compound 20 (Com20). Black arrows indicate cleavage fragments and * indicates full-length proteins. At a concentration of 200 nM GzmB produces cleavage bands, and this cleavage is prevented by both GzmB inhibitors. FIG. 14B depicts an extracellular domain schematic for β4 integrin. GzmB mediated cleavage sites identified proteomically by ATOMs (Grey arrows) fall within ligand-binding domains in the specificity-determining loop. GzmB (granzyme B); PSI (plexin-semaphorin-integrin); VWFA (von Willebrand factor A); CRR (cysteine-rich region); TM (transmembrane helix); FNIII (fibronectin-like III domain). FIG. 14C depicts β4 integrin immunostaining of healthy, bullous pemphigoid (BP), dermatitis herpetiformis (DH), and Epidermolysis Bullosa Acquisita (EBA) biopsies. Grey arrowheads indicate intact β4 integrin in areas of dermo-epidermal adhesion, black arrowheads indicate weak or absent staining. Dotted lines indicate separation between the epidermis and the dermis. β4 integrin appears to be crucial for adhesion: in the bullous pemphigoid sample, a flap of dermis in the lower right corner is attached to the epidermis and shows strong β4 staining; this area is flanked by separated epidermis with faint β4 integrin staining. Scale bars represent 200 μm.

FIGS. 15A through 15C depict GzmB-mediated collagen VII cleavage and histologic assessment in normal skin versus subepidermal blistering. FIG. 15A shows a 10% SDS-PAGE Western Blot of GzmB-mediated cleavage of collagen VII (coll VII) with and without inhibitors serpin A3N (SA3N) and Compound 20 (Com20). Black arrows indicate cleavage fragments and * indicates full-length proteins. GzmB produces cleavage bands, and this cleavage is reduced by the addition of Com20 and abolished by S3AN. FIG. 15B shows extracellular domain schematics for collagen VII. GzmB mediated cleavage sites identified proteomically by ATOMs (Grey arrows) falls in the von Willebrand factor A and fibronectin type III-2 domains, which mediate collagen VII attachment to other dermal-epidermal junction components, such as laminins and collagen IV; Coll VII, collagen VII; GzmB, granzyme B; NC, non collagenous region; CMP, cartilage matrix protein; VWFA, von Willebrand factor A; FNIII, fibronectin-like III domain; VWFA2, von Willebrand factor A 2; Pi, protein inhibitor. FIG. 15C shows Collagen VII immunostaining of healthy, bullous pemphigoid (BP), dermatitis herpetiformis (DH), and Epidermolysis Bullosa Acquisita (EBA) biopsies. Collagen VII lining is intact (Grey arrowheads) in healthy skin, but weak or absent immunoreactivity was observed in diseased samples (Black arrowheads). Dotted lines indicate separation between the epidermis and the dermis. Scale bars represent 200 rpm.

FIGS. 16A and 16B show that Collagen XVII is cleaved by GzmB and is absent in areas of epidermal separation in blistering skin biopsies. FIG. 16A shows an 8% SDS-PAGE Western Blot of GzmB-mediated cleavage of collagen XVII (coll XVII) with and without inhibitors serpin A3N (SA3N) and compound 20 (Com20). Black arrow indicates cleavage fragment and * indicates full-length protein. GzmB produces a cleavage band, and this cleavage is abolished by the addition of Com20 or S3AN. FIG. 16B shows Collagen XVII immunostaining of healthy, bullous pemphigoid (BP), dermatitis herpetiformis (DH), and Epidermolysis Bullosa Acquisita (EBA) biopsies. Collagen XVII lining is intact (Grey arrowheads) in healthy skin, but weak or absent immunoreactivity was observed in diseased samples (Black arrowheads). Dotted lines indicate separation between the epidermis and the dermis. Scale bars represent 200 rpm.

FIG. 17 demonstrates GzmB induces DEJ separation. H&E staining of healthy skin incubated for 12 hours at 37° C. in PBS, 200 nM GzmB, or 200 nM GzmB previously inactivated with 100 μM Compound 20 (Com20). Clefts between the epidermis and the dermis (black arrowheads) were observed in GzmB-treated samples but were absent in PBS control and in the sample where GzmB was inhibited by Com20. Scale bars represent 200 rpm.

FIGS. 18A through 18I provide annotated MS/MS spectra identifying neo N-terminal peptides from granzyme B cleavage sites in α4 integrin at various positions. FIG. 18A demonstrates a cleavage site at Asp110↓Asn101; FIG. 18B demonstrates a cleavage at Asp166↓Met167; FIG. 18C demonstrates a cleavage site at Asp199↓Phe200; FIG. 18D demonstrates a cleavage site at Asp302↓Ser303; FIG. 18E demonstrates a cleavage site at Asp311↓Glu312; FIG. 18F demonstrates a cleavage site at Asp358↓Val359, FIG. 18G demonstrates a cleavage site at Asp482↓Arg483; FIG. 18H demonstrates cleavage at Asp488↓Val489; and FIG. 18I demonstrates a cleavage site at Glu856↓Gln857.

FIG. 19A through 19G provides annotated MS/MS spectra identifying neo N-terminal peptides from granzyme B cleavage sites in β4 integrin at various positions. FIG. 19A demonstrates a cleavage site at Glu223↓Arg224; FIG. 19B demonstrates a cleavage site at Asp237↓Ala238; FIG. 19C demonstrates a cleavage site at Asp272↓Gly273; FIG. 19D demonstrates a cleavage site at Asp351↓Ser352;

FIG. 19E demonstrates a cleavage site at Asp442↓Gly443; FIG. 19F demonstrates a cleavage site at Asp447↓Ala448; and FIG. 19G demonstrates a cleavage site at Asp611↓Ala612.

FIG. 20 shows a 7.5% SDS-PAGE Coomassie staining of collagen I without and with the addition of 200 nM granzyme B. Black arrow indicates full length protein detected at 130 kDa, other bands represent collagen I splice variants and isoforms. Addition of granzyme B does not result in the appearance of cleavage bands. Coll I, collagen I (CollI); granzyme B (GzmB).

FIGS. 21A through 21D show annotated MS/MS spectra identifying neo N-terminal peptides from granzyme B cleavage sites in collagen VII at various positions. FIG. 21A demonstrates a cleavage site at Asp193↓Phe194, FIG. 21B demonstrates a cleavage site at Glu332↓Leu333, FIG. 21C demonstrates a cleavage site at Asp390↓Tyr391, and FIG. 21D demonstrates a cleavage site at Asp414↓Ala415.

FIG. 22A through FIG. 22C shows Compound A can prevent the cleavage of α6β4 integrin, nidogen-2 and collagen VII by granzyme B. FIG. 22A shows Coomassie staining of GzmB-mediated cleavage of α6β4 integrin with and without the granzyme B inhibitor Compound A. Recombinant human integrin α6/β4, (α6 aa 24-878, β4 aa 28-710) was incubated for 24 hours at 37° C. in 200 nM purified human GzmB. For inhibition studies, prior to the addition of substrates to the reaction, GzmB was incubated in the presence of 100 μM Compound A for 1 hour at 37° C. After 24 hours incubation, proteins were denatured, separated on a 4%-20% SDS-PAGE gel (for α6/β4). Cleavage was demonstrated using standard Coomassie staining (FIG. 22A). FIG. 22B shows a Western Blot of GzmB-mediated cleavage of nidogen-2 with and without the inhibitor Compound A. Black arrows indicate cleavage fragments and * indicates full-length protein. At a concentration of 200 nM, GzmB produces cleavage bands, and this cleavage was prevented by Compound A. Isolated nidogen-2 (amino acid residues 31-1375; 500 ng) was incubated for 24 hours at 37° C. in 200 nM purified human GzmB. For inhibition studies, prior to the addition of substrates to the reaction, GzmB was incubated in the presence of 100 μM Compound A for 1 hour at 37° C. After 24 hours incubation, nidogen-2 protein was denatured and separated on a 7.5% SDS-PAGE gel. Cleavage was detected by anti-nidogen-2 antibody (R&D Systems). FIG. 22C shows a Western Blot of GzmB-mediated cleavage of collagen VII with and without the inhibitor Compound A. Black arrows indicate cleavage fragments and * indicates full-length protein. At a concentration of 200 nM, GzmB produces cleavage bands, and this cleavage is prevented by 100 μM Compound A. Isolated collagen VII (amino acid residues 199-482; 500 ng) was incubated for 24 hours at 37° C. in 200 nM purified human GzmB. For inhibition studies, prior to the addition of substrates to the reaction, GzmB was incubated in the presence of 100 μM Compound A for 1 hour at 37° C. After 24 hours incubation, collagen VII protein was denatured and separated on a 10% SDS-PAGE gel. Cleavage was detected by anti-collagen VII antibody (ABCAM, Toronto, ON).

### DETAILED DESCRIPTION OF THE INVENTION

The present invention provides compositions and methods for the treatment of and/or prevention of skin peeling and/or blistering. The compositions can comprise formulations that include a GzmB inhibitor compound. In the methods of the invention, the composition is administered orally, topically or by systemic, subcutaneous, intradermal, or intravenous injection, and the like.

The skin consists of two main layers: the epidermis and the dermis. Blisters are accumulations of fluid within or under the dermis. Diagnosis depends on the location of the intercellular break (Clarke et al., Color Atlas of Differential Diagnosis in Dermatopathology, P. Medical Lts., Ch 4, 2014). In general, blisters can be classified into three types: 1) subcorneal (Very thin roof, breaks easily. E.g., impetigo, miliaria, staphylococcal scalded skin syndrome); 2) intra-epidermal (i.e., within the epidermis, thin roof ruptures to leave denuded surface. E.g., pemphigus, varicella, herpes simplex, acute eczema) and 3) sub-epidermal (i.e., below the epidermis, tense roof remains intact. E.g., bullous pemphigoid, dermatitis herpetiformis, erythema multiforme SJS/TEN, friction blisters). Blistering can be caused by friction, extreme temperature, chemical exposure, gas exposure, contact dermatitis, toxins, infections, crushing/pinching, autoimmunity and/or medical conditions.

Peeling refers to damage and loss of the upper layer (epidermis) of skin. Skin peeling and/or blistering can be caused by environmental factors that irritate or damage skin such as extreme temperature (hot or cold), infection, friction, sun, wind, heat, dryness and excessive humidity, repetitive irritation, chemicals, allergens, or drugs; as well as pathological conditions including autoimmune and genetic disorders.

Based on the etiology, these dermatoses are generally classified in four major groups: a) antibody-mediated, b) cutaneous adverse drug reactions, c) congenital conditions, and d) blistering caused by external insults such as burns, friction, sunlight, insect bites, infections, and chemical weapons. With respect to autoimmune skin blistering diseases, auto-antibodies are produced against structural or adhesive molecules of the skin and based on the location of the specific auto-antigens and level of blister formation, these diseases are further classified into intra-epidermal and sub-epidermal blistering diseases. Pemphigus (intra-epidermal blistering) is characterized by interepithelial blistering that are characterized by a loss of cell-cell adhesion (acantholysis) and antibodies against epithelial adhesion proteins such as desmogleins, cadherins and/or other desmosomal proteins. In sub-epidermal blistering dermatoses such as bullous pemphigoid, dermatitis herpetiformis and epidermolysis bullosa, auto-antibodies targeting components of the dermal-epidermal junction (DEJ) lead to the disruption of this basement membrane and consequent detachment of the epidermis (Baum et al., Autoimmun. Rev. 13:482-489, 2014).

As above, peeling skin syndrome (also referred to as deciduous skin, familial continuous skin peeling, exfoliative ichthyosis) refers to a group of rare inherited skin disorders characterized by painless, continual, spontaneous skin peeling (exfoliation) due to a separation of the stratum corneum (outermost layer of skin) from the underlying epidermis. Peeling skin syndromes may also exhibit blistering and/or erythema (skin reddening) and/or pruritus (itching). Symptoms of peeling skin syndromes may be observed at birth or can appear in early childhood. Two forms of peeling skin syndrome are recognized: (1) a generalized form involving the entire integument, and (2) an acral form (acral peeling skin syndrome) involving only the extremities, and mostly hands and feet. In the acral forms, patients usually develop blisters and erosions on hands and feet at birth or during infancy, which is reminiscent of the blistering skin disorder, epidermolysis bullosa simplex.

Blistering skin disorders are a group of rare skin diseases involving blistering and erosions in the skin and/or mucous membranes. Types of blistering skin diseases include: (1) autoimmune blistering diseases such as bullous pemphigoid, drug-induced pemphigus, endemic pemphigus, pemphigus erythematosus, pemphigus vegetans, pemphigus vulgaris, pemphigus foliaceus, paraneoplastic pemphigus, mucous membrane pemphigoid, epidermolysis bullosa, Linear IgA disease, bullous lupus, dermatitis herpetiformis and (2) genetic blistering diseases such as epidermolysis bullosa (EB) including for example, epidermolysis bullosa simplex, junctional epidermolysis bullosa, dystrophic epidermolysis bullosa, and epidermalysis bullosa acquisita. Sometimes, the skin will blister when it comes into contact with a cosmetic, detergent, solvent, or other chemical such as Balsam of Peru, nickel sulfate, or urushiol (poison oak, poison sumac, poison ivy). Blisters also occur due to allergic reactions caused by insect bites, extracts or stings. Chemical warfare agents (i.e., blister agents) or vesicants, can also cause large, painful blisters wherever they contact skin (e.g., mustard gas). Blistering can also occur after contact with several types of beetles that release versicants such as Cantharidin. Such blistering is associated with blistering beetle dermatitis or paederus dermatitis.

The dermal-epidermal junction (DEJ) is a specialized basement membrane between the epidermis and the dermis, which serves critical purposes for the integrity and function of the skin. It provides firm anchorage between the basal layer of the epidermis and the papillary layer of the dermis, while acting as a selective filter during cellular and molecular exchange between these two layers. Moreover, DEJ interaction with the basal layer of the epidermis determines the polarity of basal keratinocytes, maintaining proliferating cells attached to it while allowing daughter cells to migrate toward upper layers.

Structurally, the DEJ can be divided into 4 zones: 1) the basal epidermal cell membrane 2) the lamina lucida, 3) the lamina densa and 4) the fibrillar zone (papillary dermis). The first layer is represented by basal keratinocytes membrane and their specialized junctional structures called hemidesmosome, which connect keratinocytes cytoskeleton to the lamina lucida through the trans-membrane proteins α6/β4 and α3/β1 integrins, as well as collagen XVII. In the lamina lucida these proteins bridge the keratin cytoskeleton to the anchoring filaments mainly composed of laminin-5. These anchoring filaments, in turn, bind to collagen IV, the main component of the lamina densa, through nidogen and perlecan. The last junction in the DEJ is represented by the bond between elements in the lamina densa and the anchoring fibrils in the fibrillar zone. Burgeson and Christiano, Curr. Opin. Biol. 9:651-658, 1997; Aumailley and Rousselle Matrix Biol. 18(1):19-28.

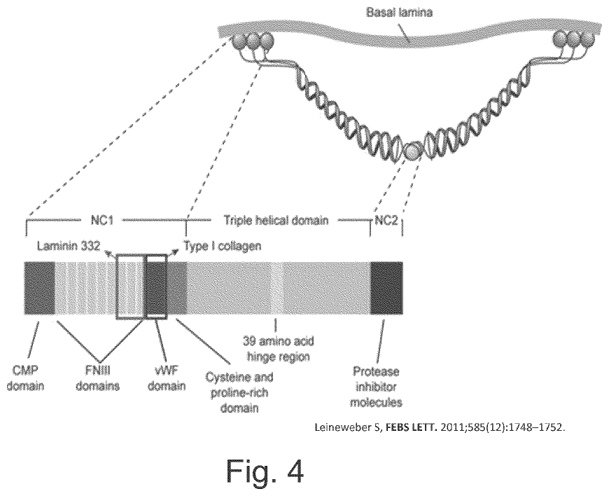

Anchoring fibrils are almost exclusively composed of Collagen VII, a non-fibrillar collagen instrumental for the structural integrity of the DEJ. Encoded by the gene COL7A1, collagen VII is a homotrimer of three al chains and consists of one central collagenous domain flanked by a C-terminal non-collagenous domain 2 (NC-2) and a N-terminal non-collagenous domain 1 (NC-1) (FIG. 4). The latter contains several subdomains with high homologies to adhesion proteins: one cartilage matrix protein like domain (CMP), nine fibronectin-like domains (FNIII), and one von-Willebrand-factor-A like domain (vWFA2). Leineweber et al., Febs Lett. 585:1748-1752, 2011. Among these domains, FNIII regions are pivotal for the interaction with several ECM components. Studies with a recombinant version of the NC1 region revealed strong binding affinity of FNIII domains with collagen I, collagen IV, laminin-5 and fibronectin. Chen et al., J. Biol. Chem. 272:14516-14522, 1997; Chen et al., J. Invest. Dermatol. 112:177-183, 1999. It is this region that allows anchorage of the papillary dermis to the lamina densa through FNIII binding to laminin-5 and collagen IV.

Due to its role as the first point of attachment between the dermis and the epidermis, any structural modification or functional impairment of collagen VII results in disruption of the DEJ with consequent epidermal detachment and blistering. Mutations of collagen VII or production of auto-antibodies against collagen VII, are the main cause of dystrophic epidermolysis bullosa and epidermolysis bullosa acquisita respectively. These conditions are characterized by extensive epidermal detachment and formation of blisters. Has, Curr. Top. Membr. 76:117-170, 2015.

GzmB is a pro-apoptotic serine protease found in the granules of cytotoxic lymphocytes (CTL) and natural killer (NK) cells. GzmB is released towards target cells, along with the pore-forming protein, perforin, resulting in its perforin-dependent internalization into the cytoplasm and subsequent induction of apoptosis (see, for e.g., Medema et al., Eur. J. Immunol. 27:3492-3498, 1997). However, GzmB can also be expressed and secreted by other types of immune (e.g., mast cell, macrophage, neutrophil, and dendritic cells) or non-immune (keratinocyte, chondrocyte) cells and has been shown to possess extracellular matrix remodeling activity (Hiebert et al., Trends Mol. Med. 18(12):732-741). In the present application, it is shown that GzmB accumulation in the skin can promote separation of the epidermis from the dermis similar to that which occurs in blistering. It is also demonstrated that GzmB cleaves key junctional proteins in the dermal epidermal junction (DEJ).

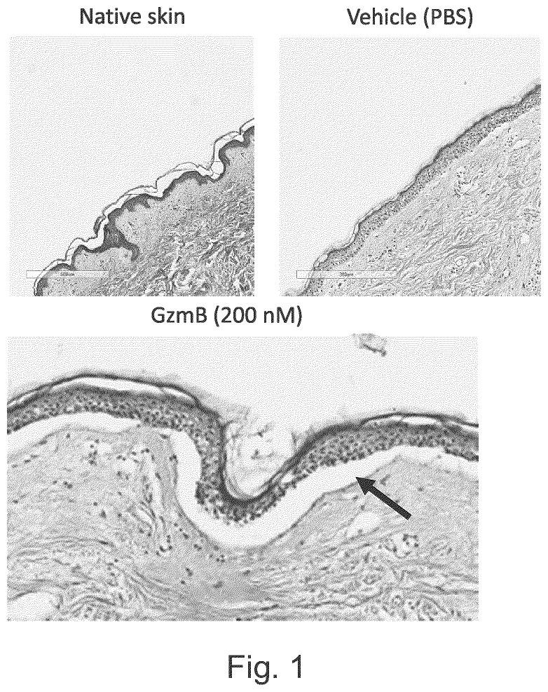

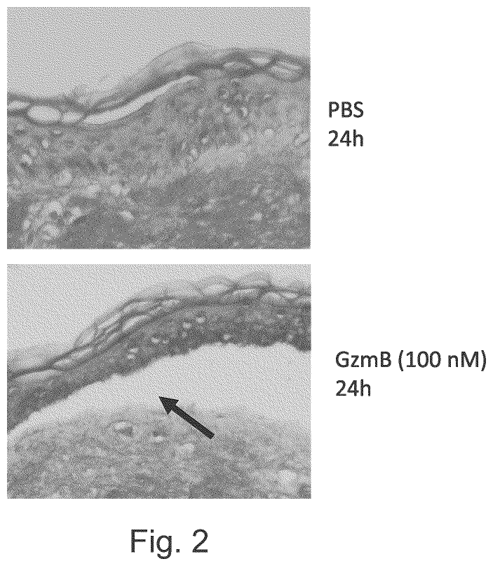

Elevated GzmB is observed in many skin diseases. In FIGS. 1 and 2 it was shown herein that the addition of GzmB to human skin induces separation of the epidermis from the dermis.

Based on the surprising results described herein, and without being bound to a theory, it is believed that the inhibition of GzmB in the skin can prevent peeling and/or blistering in skin that can be prone to peeling or blistering due to the aforementioned causes.

In one aspect, the invention provides compositions for preventing and/or treating skin peeling or blistering. The compositions comprise formulas that include a GzmB inhibitor compound or a pharmaceutically acceptable salt thereof in combination with a pharmaceutically acceptable carrier, and optionally other blister and/or wound healing ingredients or compounds.

In the practice of the invention, it has been advantageously found that the compositions of the invention can comprise certain formulations that are effective in penetration of the stratum corneum without significant complete skin penetration. The formulations of the invention are effective for intradermal delivery of the GzmB inhibitor compound rather than transdermal delivery, typically a desirable characteristic for systemic administration of a therapeutic agent. The compositions of the invention can also be formulated for intravenous or oral administration to treat certain conditions such as SJS/TEN or oral pemphigus, respectively.

GzmB Inhibitor Compounds

The formulations and methods of the invention use GzmB inhibitor compounds having Formula (I):

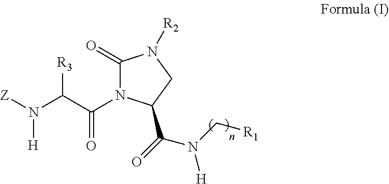

stereoisomers, tautomers, or pharmaceutically acceptable salts thereof, wherein:

R₁ is a heteroaryl group selected from

(a) 1,2,3-triazolyl, and

(b) 1,2,3,4-tetrazolyl;

n is 1 or 2;

R₂ is selected from hydrogen, C₁-C₆ alkyl, and C₃-C₆ cycloalkyl;

R₃ is selected from

(a) hydrogen,

(b) C₁-C₄ alkyl optionally substituted with a carboxylic acid, carboxylate, or carboxylate C₁-C₈ ester group (—CO₂H, —CO₂—, —C(═O)OC₁-C₈), an amide optionally substituted with an alkylheteroaryl group, or a heteroaryl group;

Z is an acyl group selected from the group

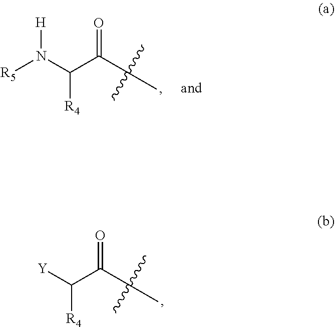

wherein

Y is hydrogen, heterocycle, —NH₂, or C₁-C₄ alkyl;

R₄ is selected from

(i) C₁-C₁₂ alkyl,

(ii) C₁-C₆ heteroalkyl optionally substituted with C₁-C₆ alkyl,

(iii) C₃-C₆ cycloalkyl,

(iv) C₆-C₁₀ aryl,

(v) heterocyclyl,

(vi) C₃-C₁₀ heteroaryl,

(vii) aralkyl, and

(viii) heteroalkylaryl;

R₅ is heteroaryl or —C(═O)—R₁₀,

wherein R₁₀ is selected from

(i) C₁-C₁₂ alkyl optionally substituted with C₆-C₁₀ aryl, C₁-C₁₀ heteroaryl, amino, or carboxylic acid,

(ii) C₁-C₁₀ heteroalkyl optionally substituted with C₁-C₆ alkyl or carboxylic acid,

(iii) C₃-C₆ cycloalkyl optionally substituted with C₁-C₆ alkyl, optionally substituted C₆-C₁₀ aryl, optionally substituted C₃-C₁₀ heteroaryl, amino, or carboxylic acid,

(iv) C₆-C₁₀ aryl optionally substituted with C₁-C₆ alkyl, optionally substituted C₆-C₁₀ aryl, optionally substituted C₃-C₁₀ heteroaryl, amino, or carboxylic acid,

(v) heterocyclyl,

(vi) C₃-C₁₀ heteroaryl,

(vii) aralkyl, and

(viii) heteroalkylaryl.

In certain embodiments, the compounds useful in the formulations and methods of the invention include compounds having Formula (I), stereoisomers, tautomers, or pharmaceutically acceptable salts thereof, wherein:

R₁ is a heteroaryl group selected from

(a) 1,2,3-triazolyl, and

(b) 1,2,3,4-tetrazolyl;

n is 1;

R₂ is selected from hydrogen, C₁-C₆ alkyl, and C₃-C₆ cycloalkyl;

R₃ is selected from

(a) hydrogen,

(b) C₁-C₄ alkyl optionally substituted with a carboxylic acid, carboxylate, or carboxylate C₁-C₈ ester group (—CO₂H, —CO₂—, —C(═O)OC₁-C₈), an amide optionally substituted with an alkylheteroaryl group, or a heteroaryl group;

Z is an acyl group selected from the group

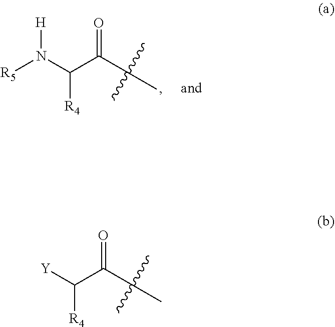

wherein R₄, R₅, and Y are as described above.

In further embodiments, the compounds useful in the formulations and methods of the invention include compounds having Formula (I), stereoisomers, tautomers, or pharmaceutically acceptable salts thereof, wherein:

R₁ is tetrazole or triazole; n is 1; R₂ is selected from hydrogen, C₁-C₆ alkyl, and C₃-C₆ cycloalkyl; R₃ is selected from hydrogen, C₁-C₄ alkyl substituted with a carboxylic acid or carboxylate group, C₁-C₄ alkyl substituted with an amide optionally substituted with an alkylheteroaryl group, or a heteroaryl group; and Z is

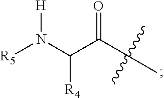

and

R₁ is tetrazole or triazole; n is 1; R₂ is selected from hydrogen, C₁-C₆ alkyl, and C₃-C₆ cycloalkyl; R₃ is independently hydrogen, or C₁-C₄ alkyl substituted with a carboxylic acid or carboxylate group, an amide optionally substituted with an alkylheteroaryl group, or a heteroaryl group; and Z is

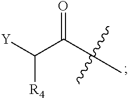

wherein

R₄ is selected from

(i) C₁-C₁₂ alkyl,

(ii) C₃-C₆ cycloalkyl,

(iii) C₆-C₁₀ aryl, and

(iv) C₃-C₁₀ heteroaryl;

R₅ is —C(═O)—R₁₀, wherein R₁₀ is selected from

(i) C₁-C₁₂ alkyl optionally substituted with C₆-C₁₀ aryl, C₁-C₁₀ heteroaryl, amino, or carboxylic acid,

(ii) C₁-C₁₀ heteroalkyl optionally substituted with C₁-C₆ alkyl or carboxylic acid,

(iii) C₃-C₆ cycloalkyl optionally substituted with C₁-C₆ alkyl, optionally substituted C₆-C₁₀ aryl, optionally substituted C₃-C₁₀ heteroaryl, amino, or carboxylic acid,

(iv) C₆-C₁₀ aryl optionally substituted with C₁-C₆ alkyl, optionally substituted C₆-C₁₀ aryl, optionally substituted C₃-C₁₀ heteroaryl, amino, or carboxylic acid,

(v) C₃-C₁₀ heteroaryl; and

Y is hydrogen, C₁-C₄ alkyl, or —NH₂.

In another embodiment, the compounds useful in the compositions and methods of the invention include compounds having Formula (II):

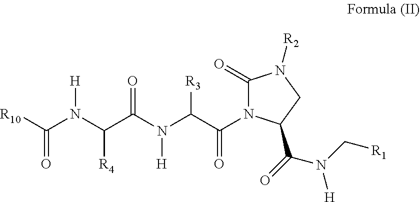

stereoisomers, tautomers, or pharmaceutically acceptable salts thereof, wherein:

R₁, R₂, R₃, R₄, and R₁₀ are as above for Formula (I).

In certain embodiments, R₁₀, when defined as C₁-C₁₂ alkyl substituted with a carboxylic acid or carboxylate group, is:

—(CH₂)ₙ—CO₂H, where n is 2, 3, 4, 5, or 6;

optionally wherein one or more single methylene carbons are substituted with a fluoro, hydroxy, amino, C₁-C₃ alkyl (e.g., methyl), or C₆-C₁₀ aryl group;

optionally wherein one or more single methylene carbons are substituted with two fluoro (e.g., difluoro, perfluoro) or C₁-C₃ alkyl (e.g., gem-dimethyl) groups;

optionally wherein one or more single methylene carbons are substituted with two alkyl groups that taken together with the carbon to which they are attached form a 3, 4, 5, or 6-membered carbocyclic ring (e.g., spiro groups such as cyclopropyl, cyclobutyl, cyclopentyl, and cyclohexyl); and

optionally wherein adjacent carbon atoms from an unsaturated carbon-carbon bond (e.g., alkenyl such as —CH═CH—) or taken form a benzene ring (e.g., 1,2-, 1,3-, and 1,4-phenylene); or

wherein R₁₀, when defined as C₃-C₆ cycloalkyl substituted with a carboxylic acid or carboxylate group, is:

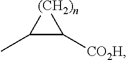

wherein n is 1, 2, 3, or 4; and optionally, for n=3 or 4, wherein adjacent carbon atoms from an unsaturated carbon-carbon bond (e.g., cyclopentenyl or cyclohexenyl).

In certain embodiments, the compounds useful in the compositions and methods of the invention include compounds having Formula (II), stereoisomers, tautomers, or pharmaceutically acceptable salts thereof, wherein:

R₁ is tetrazole or triazole;

R₂ is selected from hydrogen, C₁-C₆ alkyl, and C₃-C₆ cycloalkyl;

R₃ is hydrogen, C₁-C₄ alkyl optionally substituted with a carboxylic acid, carboxylate, or a carboxylate ester group; or C₁-C₄ alkyl optionally substituted with an amide, which may be optionally substituted with an alkylheteroaryl group;

R₄ is C₁-C₁₂ alkyl, C₃-C₆ cycloalkyl, C₆-C₁₀ aryl, C₃-C₁₀ heteroaryl, or heterocyclyl; and

R₁₀ is C₁-C₁₂ alkyl optionally substituted with C₆-C₁₀ aryl, C₁-C₁₀ heteroaryl, amino, or carboxylic acid.

In further embodiments, the compounds useful in the compositions and methods of the invention include compounds having Formula (II), stereoisomers, tautomers, or pharmaceutically acceptable salts thereof, wherein:

R₁ is tetrazole or triazole;

R₂ is selected from hydrogen, C₁-C₆ alkyl, and C₃-C₆ cycloalkyl;

R₃ is hydrogen, C₁-C₄ alkyl optionally substituted with a carboxylic acid, carboxylate, or a carboxylate ester group;

R₄ is C₁-C₈ alkyl or C₃-C₆ cycloalkyl; and

R₁₀ is selected from:

(a) C₁-C₃ alkyl substituted with C₆-C₁₀ aryl (e.g., phenyl) or C₁-C₁₀ heteroaryl (e.g., triazolyl or tetrazolyl);

(b) —(CH₂)ₙ—CO₂H, where n is 2, 3, 4, 5, or 6;

(c)

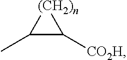

wherein n is 1, 2, 3, or 4.

In one embodiment, the compounds useful in the compositions and methods of the invention include compounds having Formula (II), stereoisomers, tautomers, or pharmaceutically acceptable salts thereof, wherein:

R₁ is tetrazole;

R₂ is selected from hydrogen, C₁-C₆ alkyl (e.g., methyl), and C₃-C₆ cycloalkyl (e.g., cyclohexyl);

R₃ is hydrogen or C₁-C₄ alkyl optionally substituted with a carboxylic acid, carboxylate, or a carboxylate ester group (e.g., C₂ alkyl substituted with a carboxylic acid, carboxylate, or a carboxylate ester group);

R₄ is C₁-C₈ alkyl (e.g., C₄ alkyl); and

R₁₀ is —(CH₂)ₙ—CO₂H, where n is 2, 3, 4, 5, or 6 (e.g., —(CH₂)ₙ—CO₂H, where n is 2).

Representative compounds of Formula (II) include C₁-C₅.

In a further embodiment, the compounds useful in the compositions and methods of the invention include compounds having Formula (III):

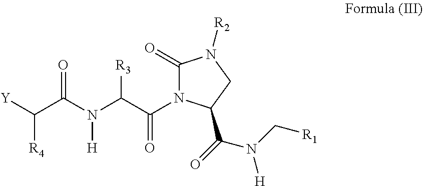

stereoisomers, tautomers, or pharmaceutically acceptable salts thereof, wherein R₁, R₂, R₃, R₄, and Y are as defined above for Formula (I).

In certain embodiments, the compounds useful in the compositions and methods of the invention include compounds having Formula (III), stereoisomers, tautomers, or pharmaceutically acceptable salts thereof, wherein:

R₁ is tetrazole or triazole;

R₂ is selected from hydrogen, C₁-C₆ alkyl, and C₃-C₆ cycloalkyl;

R₃ is hydrogen; C₁-C₄ alkyl optionally substituted with a carboxylic acid, carboxylate, or a carboxylate ester group; or C₁-C₄ alkyl optionally substituted with an amide, which may be optionally substituted with an alkylheteroaryl group;

R₄ is C₁-C₁₂ alkyl, C₃-C₆ cycloalkyl, C₆-C₁₀ aryl, C₃-C₁₀ heteroaryl, or heterocyclyl; and

Y is hydrogen, C₁-C₄ alkyl, or —NH₂.

In further embodiments, the compounds useful in the compositions and methods of the invention include compounds having Formula (III), stereoisomers, tautomers, or pharmaceutically acceptable salts thereof, wherein:

R₁ is tetrazole or triazole;

R₂ is selected from hydrogen, C₁-C₆ alkyl, and C₃-C₆ cycloalkyl;

R₃ is C₁-C₄ alkyl optionally substituted with a carboxylic acid, carboxylate, or a carboxylate ester group;

R₄ is selected from

(i) C₁-C₈ alkyl (e.g., methyl, ethyl, n-propyl, i-propyl),

(ii) C₃-C₆ cycloalkyl (i.e., cyclopropyl, cyclobutyl, cyclopentyl, cyclohexyl),

(iii) C₆-C₁₀ aryl (e.g., phenyl),

(iv) C₃-C₁₀ heteroaryl (e.g., thiophenyl), and

(v) heterocyclyl (e.g., morpholinyl); and

Y is hydrogen.

Representative compounds of Formula (III) include C₆.

For the compounds of Formulae (I), (II), or (III), representative substituents R₃ include the following:

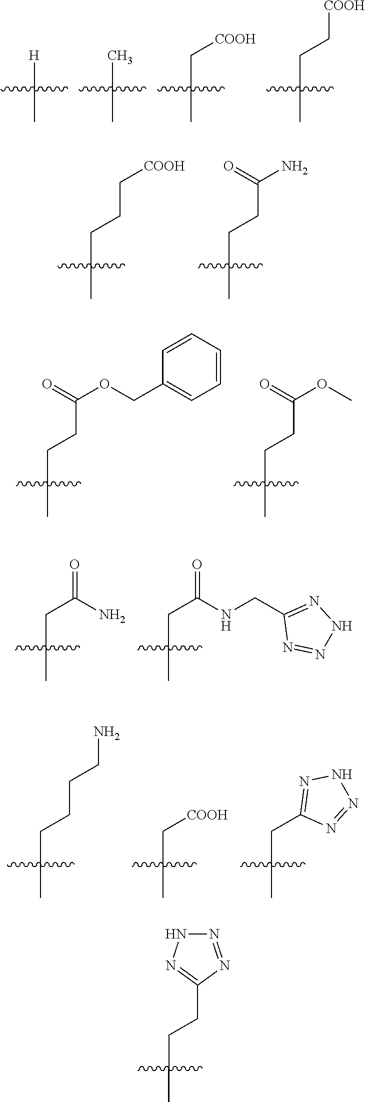

For the compounds of Formulae (I), (II), or (III), representative substituents R₄ include the following:

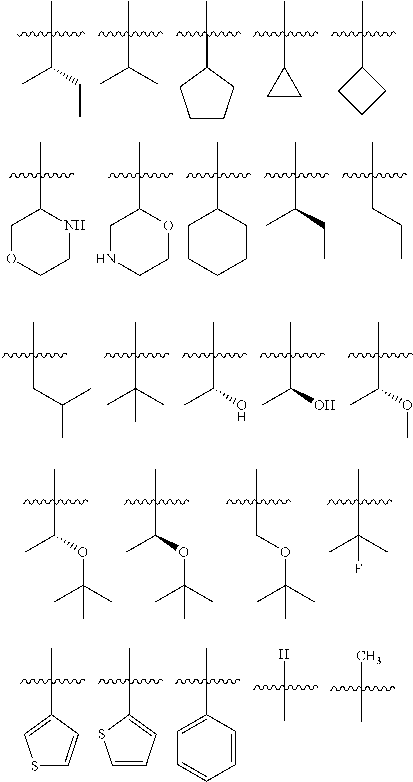

For the compounds of Formulae (I), (II), or (III), representative substituents R₅ include the following:

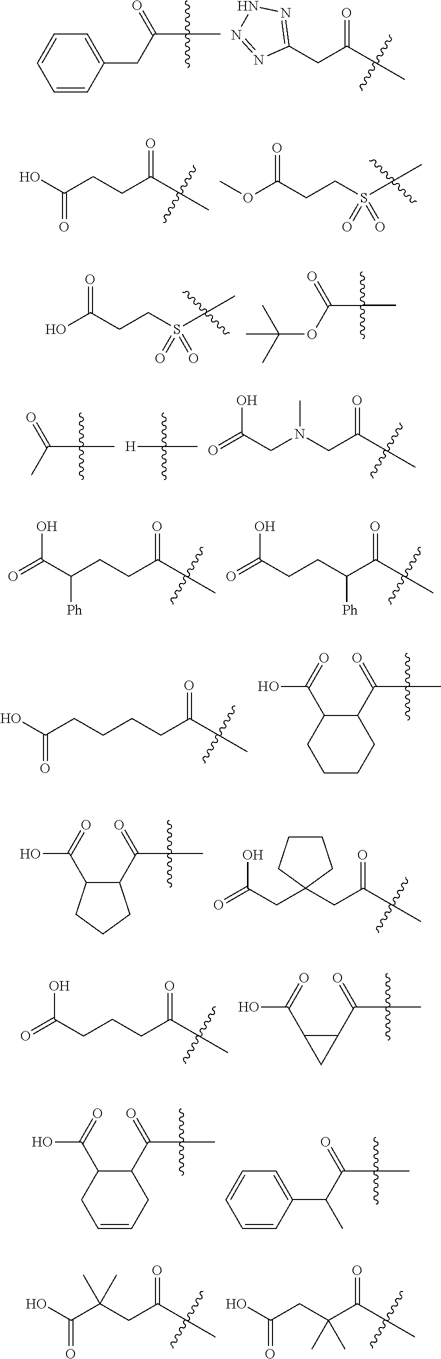

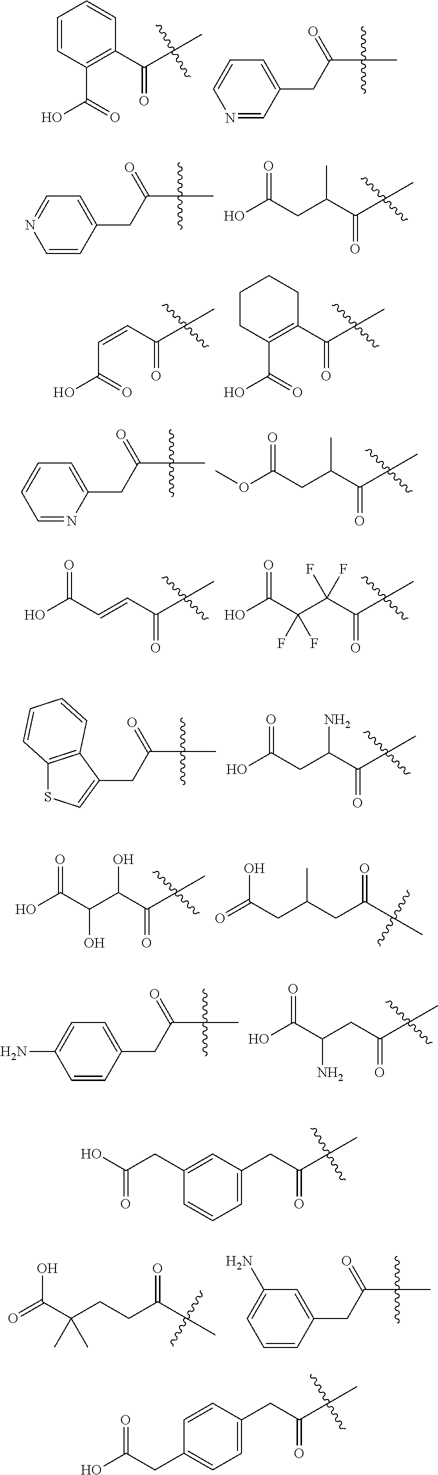

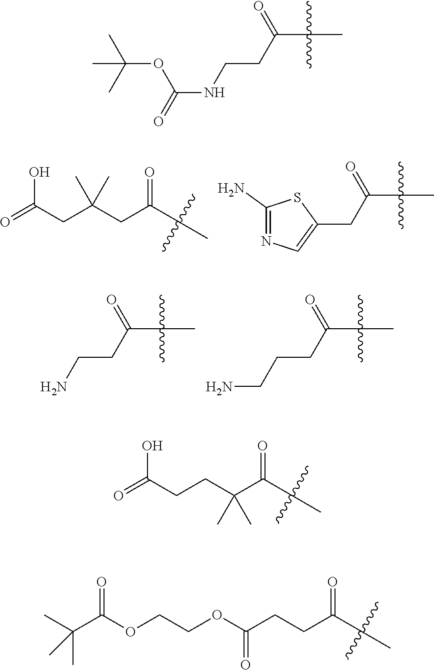

Each of the inhibitor compounds contain asymmetric carbon centers and give rise to stereoisomers (i.e., optical isomers such as diastereomers and enantiomers). It will be appreciated that the present invention includes such diastereomers as well as their racemic and resolved enantiomerically pure forms. It will also be appreciated that in certain configurations, the relative stereochemistry of certain groups may be depicted as “cis” or “trans” when absolute stereochemistry is not shown.

Some of the compounds described herein contain olefinic double bonds, and unless specified otherwise, are meant to include both E and Z geometric isomers.

Certain of the compounds may exist in one or more tautomeric forms (e.g., acid or basic forms depending on pH environment). It will be appreciated that the compounds include their tautomeric forms (i.e., tautomers).

When the compounds are basic, salts may be prepared from pharmaceutically acceptable non-toxic acids, including inorganic and organic acids. Examples of such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, and p-toluenesulfonic acids.

The following definitions unless otherwise indicated.

As used herein, the term “alkyl” refers to a saturated or unsaturated, branched, straight-chain or cyclic monovalent hydrocarbon group derived by the removal of one hydrogen atom from a single carbon atom of a parent alkane, alkene, or alkyne. Representative alkyl groups include methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl (allyl), cycloprop-1-en-1-yl; cycloprop-2-en-1-yl, prop-1-yn-1-yl, and prop-2-yn-1-yl; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, and but-3-yn-1-yl; and the like. Where a specific level of saturation is intended, the expressions “alkanyl,” “alkenyl,” and “alkynyl” are used. Alkyl groups include cycloalkyl groups. The term “cycloalkyl” refers to mono-, bi-, and tricyclic alkyl groups having the indicated number of carbon atoms. Representative cycloalkyl groups include cyclopropyl, cyclopentyl, cycloheptyl, adamantyl, cyclododecylmethyl, and 2-ethyl-1-bicyclo[4.4.0]decyl groups. The alkyl group may be unsubstituted or substituted as described below.

“Alkanyl” refers to a saturated branched, straight-chain, or cyclic alkyl group. Representative alkanyl groups include methanyl; ethanyl; propanyls such as propan-1-yl, propan-2-yl(isopropyl), and cyclopropan-1-yl; butanyls such as butan-1-yl, butan-2-yl (sec-butyl), 2-methyl-propan-1-yl(isobutyl), 2-methyl-propan-2-yl(t-butyl), and cyclobutan-1-yl; and the like. The alkanyl group may be substituted or unsubstituted. Representative alkanyl group substituents include

—R₁₄, —OR₁₄, —SR₁₄, —NR₁₄(R₁₅),

—X, —CX₃, —CN, —NO₂,

—C(═O)R₁₄, —C(═O)OR₁₄, —C(═O)NR₁₄(R₁₅), —C(═O)SR₁₄,

—C(═NR₁₄)R₁₄, —C(═NR₁₄)OR₁₄, —C(═NR₁₄)NR₁₄(R₁₅), —C(═NR₁₄)SR₁₄,

—C(═S)R₁₄, —C(═S)OR₁₄, —C(═S)NR₁₄(R₁₅), —C(═S)SR₁₄,

—NR₁₄C(═O)NR₁₄(R₁₅), —NR₁₄(═NR₁₄)NR₁₄(R₁₅), —NR₁₄C(═S)NR₁₄(R₁₅),

—S(═O)₂R₁₄, —S(═O)₂OR₁₄, —S(═O)₂NR₁₄(R₁₅),

—OC(═O)R₁₄, —OC(═O)OR₁₄, —OC(═O)NR₁₄(R₁₅), —OC(═O)SR₁₄,

—OS(═O)₂OR₁₄, —OS(═O)₂NR₁₄(R₁₅), and

—OP(═O)₂(OR₁₄),

wherein each X is independently a halogen; and R₁₄ and R₁₅ are independently hydrogen, C₁-C₆ alkyl, C₆-C₁₄ aryl, arylalkyl, C₃-C₁₀ heteroaryl, and heteroarylalkyl, as defined herein.

In certain embodiments, two hydrogen atoms on a single carbon atom can be replaced with ═O, ═NR₁₂, or ═S.

“Alkenyl” refers to an unsaturated branched, straight-chain, cyclic alkyl group, or combinations thereof having at least one carbon-carbon double bond derived by the removal of one hydrogen atom from a single carbon atom of a parent alkene. The group may be in either the cis or trans conformation about the double bond(s). Representative alkenyl groups include ethenyl; propenyls such as prop-1-en-1-yl, prop-1-en-2-yl, prop-2-en-1-yl (allyl), prop-2-en-2-yl, and cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, and cyclobuta-1,3-dien-1-yl; and the like. The alkenyl group may be substituted or unsubstituted. Representative alkenyl group substituents include

—R₁₄,

—X, —CX₃, —CN,

—C(═O)R₁₄, —C(═O)OR₁₄, —C(═O)NR₁₄(R₁₅), —C(═O)SR₁₄,

—C(═NR₁₄)R₁₄, —C(═NR₁₄)OR₁₄, —C(═NR₁₄)NR₁₄(R₁₅), —C(═NR₁₄)SR₁₄,

—C(═S)R₁₄, —C(═S)OR₁₄, —C(═S)NR₁₄(R₁₅), —C(═S)SR₁₄,

wherein each X is independently a halogen; and R₁₄ and R₁₅ are independently hydrogen, C₁-C₆ alkyl, C₆-C₁₄ aryl, arylalkyl, C₃-C₁₀ heteroaryl, and heteroarylalkyl, as defined herein.

“Alkynyl” refers to an unsaturated branched, straight-chain, or cyclic alkyl group having at least one carbon-carbon triple bond derived by the removal of one hydrogen atom from a single carbon atom of a parent alkyne. Representative alkynyl groups include ethynyl; propynyls such as prop-1-yn-1-yl and prop-2-yn-1-yl; butynyls such as but-1-yn-1-yl, but-1-yn-3-yl, and but-3-yn-1-yl; and the like. The alkynyl group may be substituted or unsubstituted. Representative alkynyl group substituents include those as described above for alkenyl groups.

The term “haloalkyl” refers to an alkyl group as defined above having the one or more hydrogen atoms replaced by a halogen atom. Representative haloalkyl groups include halomethyl groups such as chloromethyl, fluoromethyl, and trifluoromethyl groups; and haloethyl groups such as chloroethyl, fluoroethyl, and perfluoroethyl groups. The term “heteroalkyl” refers to an alkyl group having the indicated number of carbon atoms and where one or more of the carbon atoms is replaced with a heteroatom selected from O, N, or S. Where a specific level of saturation is intended, the expressions “heteroalkanyl,” “heteroalkenyl,” and “heteroalkynyl” are used. Representative heteroalkyl groups include ether, amine, and thioether groups. Heteroalkyl groups include heterocyclyl groups. The term “heterocyclyl” refers to a 5- to 10-membered non-aromatic mono- or bicyclic ring containing 1-4 heteroatoms selected from O, S, and N. Representative heterocyclyl groups include pyrrolidinyl, piperidinyl, piperazinyl, tetrahydrofuranyl, tetrahydropuranyl, and morpholinyl groups. The heteroalkyl group may be substituted or unsubstituted. Representative heteroalkyl substituents include

—R₁₄, —OR₁₄, —SR₁₄, —NR₁₄(R₁₅),

—X, —CX₃, —CN, —NO₂,

—C(═O)R₁₄, —C(═O)OR₁₄, —C(═O)NR₁₄(R₁₅), —C(═O)SR₁₄,

—C(═NR₁₄)R₁₄, —C(═NR₁₄)OR₁₄, —C(═NR₁₄)NR₁₄(R₁₅), —C(═NR₁₄)SR₁₄,

—C(═S)R₁₄, —C(═S)OR₁₄, —C(═S)NR₁₄(R₁₅), —C(═S)SR₁₄,

—NR₁₄C(═O)NR₁₄(R₁₅), —NR₁₄(═NR₁₄)NR₁₄(R₁₅), —NR₁₄C(═S)NR₁₄(R₁₅),

—S(═O)₂R₁₄, —S(═O)₂OR₁₄, —S(═O)₂NR₁₄(R₁₅),

—OC(═O)R₁₄, —OC(═O)OR₁₄, —OC(═O)NR₁₄(R₁₅), —OC(═O)SR₁₄,

—OS(═O)₂OR₁₄, —OS(═O)₂NR₁₄(R₁₅), and

—OP(═O)₂(OR₁₄),

wherein each X is independently a halogen; and R₁₄ and R₁₅ are independently hydrogen, C₁-C₆ alkyl, C₆-C₁₄ aryl, arylalkyl, C₃-C₁₀ heteroaryl, and heteroarylalkyl, as defined herein.

In certain embodiments, two hydrogen atoms on a single carbon atom can be replaced with ═O, ═NR₁₂, or ═S.

The term “alkoxy” refers to an alkyl group as described herein bonded to an oxygen atom. Representative C₁-C₃ alkoxy groups include methoxy, ethoxy, propoxy, and isopropoxy groups.

The term “alkylamino” refers an alkyl group as described herein bonded to a nitrogen atom. The term “alkylamino” includes monoalkyl- and dialkylaminos groups. Representative C₁-C₆ alkylamino groups include methylamino, dimethylamino, ethylamino, methylethylamino, diethylamino, propylamino, and isopropylamino groups.

The term “alkylthio” refers an alkyl group as described herein bonded to a sulfur atom. Representative C₁-C₆ alkylthio groups include methylthio, propylthio, and isopropylthio groups.

The term “aryl” refers to a monovalent aromatic hydrocarbon group derived by the removal of one hydrogen atom from a single carbon atom of a parent aromatic ring system. Suitable aryl groups include groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In certain embodiments, the aryl group is a C₅-C₁₄ aryl group. In other embodiments, the aryl group is a C₅-C₁₀ aryl group. The number of carbon atoms specified refers to the number of carbon atoms in the aromatic ring system. Representative aryl groups are phenyl, naphthyl, and cyclopentadienyl. The aryl group may be substituted or unsubstituted. Representative aryl group substituents include

—R₁₄, —OR₁₄, —SR₁₄, —NR₁₄(R₁₅),

—X, —CX₃, —CN, —NO₂,

—C(═O)R₁₄, —C(═O)OR₁₄, —C(═O)NR₁₄(R₁₅), —C(═O)SR₁₄,

—C(═NR₁₄)R₁₄, —C(═NR₁₄)OR₁₄, —C(═NR₁₄)NR₁₄(R₁₅), —C(═NR₁₄)SR₁₄,

—C(═S)R₁₄, —C(═S)OR₁₄, —C(═S)NR₁₄(R₁₅), —C(═S)SR₁₄,

—NR₁₄C(═O)NR₁₄(R₁₅), —NR₁₄(═NR₁₅)NR₁₄(R₁₅), —NR₁₄C(═S)NR₁₄(R₁₅),

—S(═O)₂R₁₄, —S(═O)₂OR₁₄, —S(═O)₂NR₁₄(R₁₅),

—OC(═O)R₁₄, —OC(═O)OR₁₄, —OC(═O)NR₁₄(R₁₅), —OC(═O)SR₁₄,

—OS(═O)₂OR₁₄, —OS(═O)₂NR₁₄(R₁₅), and

—OP(═O)₂(OR₁₄),

wherein each X is independently a halogen; and R₁₄ and R₁₅ are independently hydrogen, C₁-C₆ alkyl, C₆-C₁₄ aryl, arylalkyl, C₃-C₁₀ heteroaryl, and heteroarylalkyl, as defined herein.

The term “aralkyl” refers to an alkyl group as defined herein with an aryl group, optionally substituted, as defined herein substituted for one of the alkyl group hydrogen atoms. Suitable aralkyl groups include benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl, and the like. Where specific alkyl moieties are intended, the terms aralkanyl, aralkenyl, and aralkynyl are used. In certain embodiments, the aralkyl group is a C₆-C₂₀ aralkyl group, (e.g., the alkanyl, alkenyl, or alkynyl moiety of the aralkyl group is a C₁-C₆ group and the aryl moiety is a C₅-C₁₄ group). In other embodiments, the aralkyl group is a C₆-C₁₃ aralkyl group (e.g., the alkanyl, alkenyl, or alkynyl moiety of the aralkyl group is a C₁-C₃ group and the aryl moiety is a C₅-C₁₀ aryl group. In certain embodiments, the aralkyl group is a benzyl group.

The term “heteroaryl” refers to a monovalent heteroaromatic group derived by the removal of one hydrogen atom from a single atom of a parent heteroaromatic ring system, which may be monocyclic or fused ring (i.e., rings that share an adjacent pair of atoms). A “heteroaromatic” group is a 5- to 14-membered aromatic mono- or bicyclic ring containing 1-4 heteroatoms selected from O, S, and N. Representative 5- or 6-membered aromatic monocyclic ring groups include pyridine, pyrimidine, pyridazine, furan, thiophene, thiazole, oxazole, and isooxazole. Representative 9- or 10-membered aromatic bicyclic ring groups include benzofuran, benzothiophene, indole, pyranopyrrole, benzopyran, quionoline, benzocyclohexyl, and naphthyridine. Suitable heteroaryl groups include groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In certain embodiments, the heteroaryl group is a 5-14 membered heteroaryl group. In other embodiments, the heteroaryl group is a 5-10 membered heteroaryl group. Preferred heteroaryl groups are those derived from thiophene, pyrrole, benzothiophene, benzofuran, indole, pyridine, quinoline, imidazole, oxazole, and pyrazine. The heteroaryl group may be substituted or unsubstituted. Representative heteroaryl group substituents include those described above for aryl groups.

The term “heteroarylalkyl” refers to an alkyl group as defined herein with a heteroaryl group, optionally substituted, as defined herein substituted for one of the alkyl group hydrogen atoms. Where specific alkyl moieties are intended, the terms heteroarylalkanyl, heteroarylalkenyl, or heteroarylalkynyl are used. In certain embodiments, the heteroarylalkyl group is a 6-20 membered heteroarylalkyl (e.g., the alkanyl, alkenyl or alkynyl moiety of the heteroarylalkyl is a C₁-C₆ group and the heteroaryl moiety is a 5-14-membered heteroaryl group. In other embodiments, the heteroarylalkyl group is a 6-13 membered heteroarylalkyl (e.g., the alkanyl, alkenyl or alkynyl moiety is C₁-C₃ group and the heteroaryl moiety is a 5-10-membered heteroaryl group).

The term “acyl” group refers to the —C(═O)—R′ group, where R′ is selected from optionally substituted alkyl, optionally substituted aryl, and optionally substituted heteroaryl, as defined herein.

The term “halogen” or “halo” refers to fluoro, chloro, bromo, and iodo groups.

The term “substituted” refers to a group in which one or more hydrogen atoms are each independently replaced with the same or different substituent(s).

The term “pharmaceutically acceptable salts” refers to salts prepared from pharmaceutically acceptable bases including inorganic bases and organic bases. Representative salts derived from inorganic bases include aluminum, ammonium, calcium, copper, ferric, ferrous, lithium, magnesium, manganic, manganous, ammonium, potassium, sodium, and zinc salts. Representative salts derived from pharmaceutically acceptable organic bases include salts of primary, secondary and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, and trimethamine.

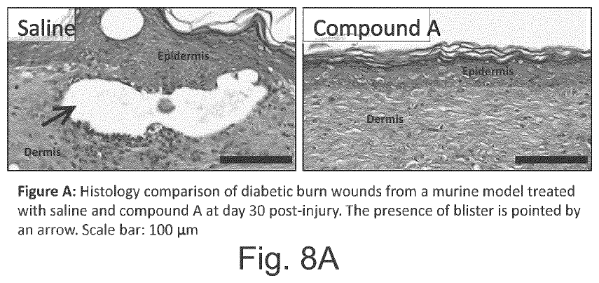

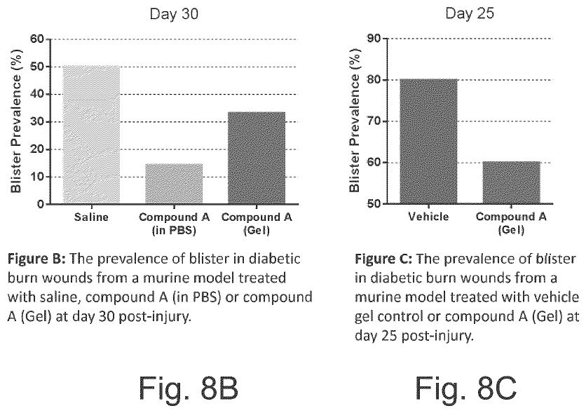

Representative compounds and related intermediates were prepared from commercially available starting materials or starting materials prepared by conventional synthetic methodologies. Representative compounds were prepared according to Methods A to C as described below and illustrated in FIGS. 8-10. The preparations of certain intermediates (I-1 to I-4) useful in the preparation of compounds of the invention are described in WO 2017/132771 (incorporated herein by reference in its entirety).

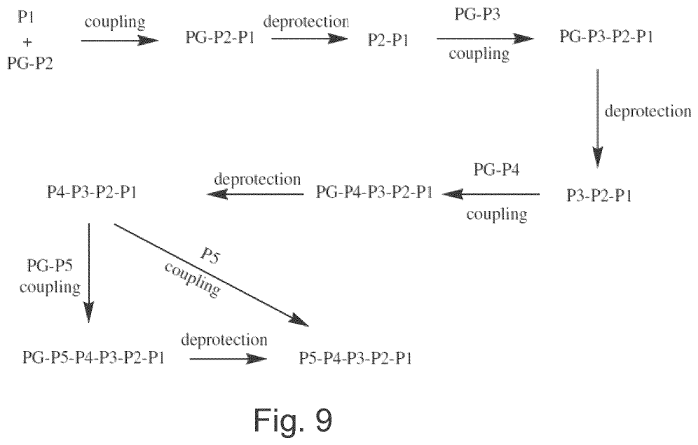

FIGS. 9-11 present schematic illustrations of representative synthetic pathways for the preparation of representative compounds of the invention P5-P4-P3-P2-P1. As used herein, “P5-P4-P3-P2-P1” refers to compounds of the invention prepared from five (5) components: P1, P2, P3, P4, and P5. Protected version of the components useful in the preparation of the compounds of the invention are designated as, for example, “PG-P2,” “PG-P2-P1,” “PG-P3,” and “PG-P3-P2-P1,” where “PG” is refers to a protecting group that allows for the coupling of, for example, P1 to P2 or P3 to P1-P2, and that is ultimately removed to provide, for example, P1-P2 or P1-P2-P3.

FIG. 9 is a schematic illustration of another representative synthetic pathway for the preparation of representative compounds of the invention P5-P4-P3-P2-P1 starting from P5. In this pathway, compound P5-P4-P3-P2-P1 is prepared in a stepwise manner starting with P5 by sequential coupling steps, separated as appropriate by deprotection steps and other chemical modifications. As shown in FIG. 9, P5 is coupled with PG-P4 to provide P5-P4-PG, which is then deprotected to provide P5-P4 and ready for coupling with the next component, P3-PG. The process is continued with subsequent couplings PG-P2 with P5-P4-P3 and PG-P1 with P5-P4-P3-P2 to ultimately provide P5-P4-P3-P2-P1.

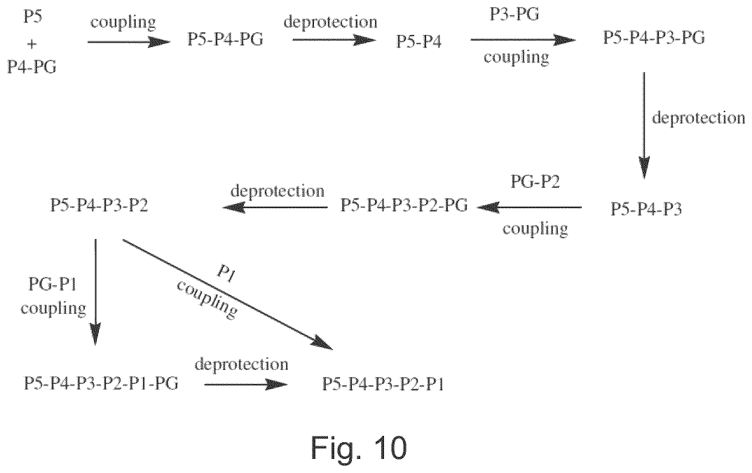

FIG. 10 is a schematic illustration of a representative synthetic pathway for the preparation of representative compounds of the invention P5-P4-P3-P2-P1 starting from P1. In this pathway, compound P5-P4-P3-P2-P1 is prepared in a stepwise manner starting with P1 by sequential coupling steps, separated as appropriate by deprotection steps and other chemical modifications. As shown in FIG. 10, P1 is coupled with PG-P2 to provide PG-P2-P1, which is then deprotected to provide P2-P1 and ready for coupling with the next component, PG-P3. The process is continued with subsequent couplings PG-P4 with P3-P2-P1 and PG-P5 with P4-P3-P2-P1 to ultimately provide P5-P4-P3-P2-P1.

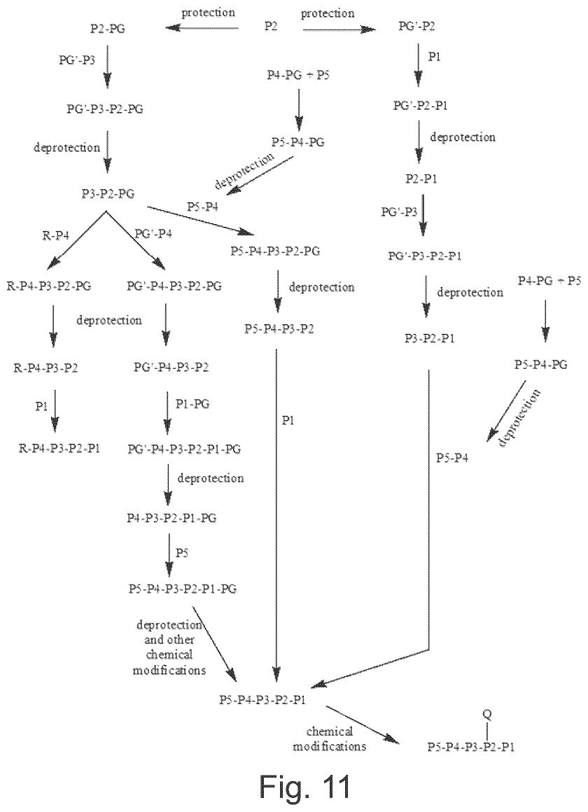

FIG. 11 is a schematic illustration of a further representative synthetic pathway for the preparation of representative compounds of the invention P5-P4-P3-P2-P1 starting from a component other than P1 or P5. In this pathway, compound P5-P4-P3-P2-P1 is prepared in a stepwise manner starting with P2 by sequential coupling steps, separated as appropriate by deprotection steps and other chemical modifications. As shown in FIG. 11, there are multiple pathways to P5-P4-P3-P2-P1. Examples C₁-C₆ recited below were prepared by this method.

The preparation of representative compounds and their characterization are described in Examples C₁-C₆ of WO 2017/132771 (incorporated herein by reference in its entirety). The structures of representative compounds are set forth in Table 1.

TABLE 1
Representative Compounds.

| Cmpd # | Structure |
|---|---|
| C1 | 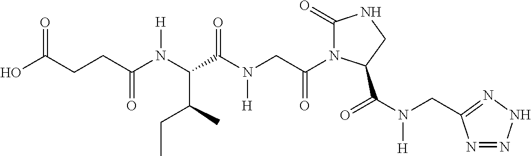 [SMILES: CC[C@H](C)[C@H](NC(=O)CCC(=O)O)C(=O)NCC(=O)N1C(=O)NC[C@H]1C(=O)NCc1nn[nH]n1] |
| C2 | 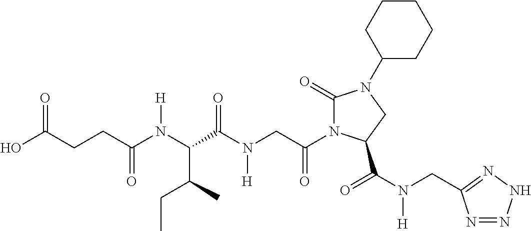 [SMILES: CC[C@H](C)[C@H](NC(=O)CCC(=O)O)C(=O)NCC(=O)N1C(=O)N(C2CCCCC2)C[C@H]1C(=O)NCc1nn[nH]n1] |
| C3 | 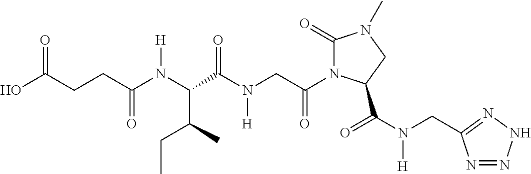 [SMILES: CC[C@H](C)[C@H](NC(=O)CCC(=O)O)C(=O)NCC(=O)N1C(=O)N(C)C[C@H]1C(=O)NCc1nn[nH]n1] |
| C4 | 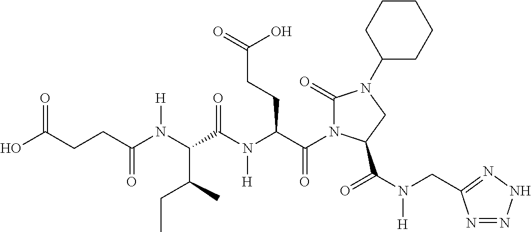 [SMILES: CC[C@H](C)[C@H](NC(=O)CCC(=O)O)C(=O)N[C@@H](CCC(=O)O)C(=O)N1C(=O)N(C2CCCCC2)C[C@H]1C(=O)NCc1nn[nH]n1] |
| C5 | 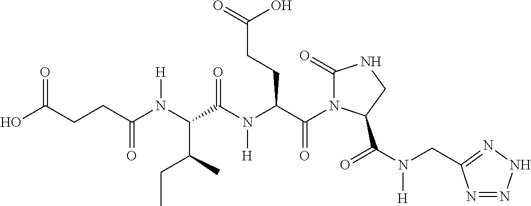 [SMILES: CC[C@H](C)[C@H](NC(=O)CCC(=O)O)C(=O)N[C@@H](CCC(=O)O)C(=O)N1C(=O)NC[C@H]1C(=O)NCc1nn[nH]n1] |
| C6 | 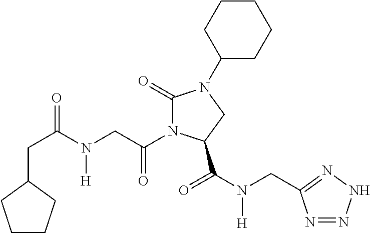 [SMILES: O=C(CC1CCCC1)NCC(=O)N1C(=O)N(C2CCCCC2)C[C@H]1C(=O)NCc1nn[nH]n1] |

The compounds identified in Table 1 exhibited GzmB inhibitory activity. In certain embodiments, select compounds exhibited IC₅₀<50,000 nM. In other embodiments, select compounds exhibited IC₅₀<10,000 nM. In further embodiments, select compounds exhibited IC₅₀<1,000 nM. In still further embodiments, select compounds exhibited IC₅₀<100 nM. In certain embodiments, select compounds exhibited IC₅₀ from 10 nM to 100 nM, preferably from 1 nM to 10 nM, more preferably from 0.1 nM to 1 nM, and even more preferably from 0.01 nM to 0.1 nM.

None of the compounds demonstrated an ability to significantly inhibit any of the caspases evaluated at a concentration of 50 μM. In certain embodiments, the compounds exhibited less than 50% inhibition at 50 μM. In other embodiments, the compounds exhibited greater than 50% inhibition at 50 μM, but less than 10% inhibition at 25 μM.

The results demonstrate that select compounds selectively inhibit GzmB without significantly inhibiting caspases.

Compositions

As noted above, the compositions of the invention include a GzmB inhibitor as described herein. A representative GzmB inhibitor compound useful in these compositions is 4-(((2S,3S)-1-((2-((S)-5-(((2H-tetrazol-5-yl)methyl)carbamoyl)-3-cyclohexyl-2-oxoimidazolidin-1-yl)-2-oxoethyl)amino)-3-methyl-1-oxopentan-2-yl)amino)-4-oxobutanoic acid (referred to herein as Compound A), and pharmaceutically acceptable salts thereof.

In certain embodiments, the GzmB inhibitor (e.g., Compound A, Compound 20 or Serpin A3N, and the like) is present in the formulation in an amount from about 0.25 to about 25.0 mg/mL of the formulation. In certain embodiments, the GzmB inhibitor is present in an amount from about 3.0 to about 15 mg/mL of the formulation. In other embodiments, the GzmB inhibitor is present in an amount from about 10.0 to about 15.0 mg/mL of the formulation. In one embodiment, the GzmB inhibitor is present in about 10.0 mg/mL of the formulation.

The pH of the formulations of the invention can be readily varied as desired by adjustment with, for example, a base such a triethanol amine. In certain embodiments, the formulation pH is from about 4.0 to about 7.4. In other embodiments, the formulation pH is from about 4.0 to about 6.5. In other embodiments, such as for topical application to the skin, the formulation pH is about 6.0.

In certain embodiments, the formulations of the invention are aqueous formulations that also include organic components. The aqueous formulations are buffered and have a pH in the range from about 4 to about 7, including from about 4 to 5, 4 to 7, and 5 to 7. In certain embodiments, the pH is from about 4.0 to about 6.5. In other embodiments, the pH is about 6.0. Suitable buffers include those useful for pharmaceutical and cosmetic compositions that are topically administered or administered by injection. Representative buffers include acetate and phosphate buffers.

It has been previously determined that the GzmB inhibitor skin permeability decreases when the pH of the formulation increases.

In addition to the GzmB inhibitor compound, the formulations of the invention can include one or more penetration enhancer. A suitable penetration enhancer can include propylene glycol (PG), urea, Tween 80, dimethyl isosorbide (DMI), Transcutol, N-methyl-2-pyrollidone (MNP), and the like. The amount of penetration enhancer can be carried to achieve the desired formulation properties.

A representative penetration enhancer is propylene glycol (PG). The amount of propylene glycol present in the formulation can range from about 5 to 80 percent by weight based on the total weight of the formulation. In certain embodiments, propylene glycol is present in an amount from about 15 to about 25 percent by weight based on the total weight of the formulation. In other embodiments, propylene glycol is present in an amount about 20 percent by weight based on the total weight of the formulation. For certain topical applications, propylene glycol can be used in an amount up to about 80% w/w.

It will be appreciated that suitable polyols other than propylene glycol can be used in the formulations. Propylene glycol or other suitable polyols provide for hydrogel formulation and prevent rapid drying of the gel. Compared to other polyols, such as glycerin, propylene glycol offers the advantage of being a penetration enhancer and also a better solvent or co-solvent.

Representative formulations of the invention include a GzmB inhibitor (0.5 to 15 mg/mL), a penetration enhancer (propylene glycol, 15 to 25 percent by weight), and an aqueous acetate buffer at pH 5.

In certain embodiments, the formulation further includes one or more viscosity enhancers or gelling agents. A suitable viscosity enhancer includes Carbopols, Carbomers, carboxymethyl cellulose (CMC), starches, vegetable gums, sugars, and the like.

A representative viscosity enhancer can include a crosslinked polyacrylate polymer, such as a polyacrylate polymer crosslinked with an ether of pentaerythritol (e.g., Carbopol® 940 or 980). The viscosity enhancer is typically present in the formulation in an amount from about 0.1 to about 5.0 percent by weight based on the total weight of the formulation (e.g., 0.5 percent by weight based on the total weight of the formulation).

When Carbopol® 940 or 980 is used, the formulation containing less than about 0.5% w/w is a lotion rather than a gel, and at a pH of less than about 5, the formulation is not viscous.

For a formulation that includes Carbopol® 940 or 980, which is pH sensitive, the pH is from about 5 and about 6 to obtain the formulation as a gel. The final pH of the formulation can be adjusted to achieve the desired pH range by using a suitable base as the pharmaceutically acceptable base (e.g., sodium hydroxide, triethylamine, or triethanolamine, and the like). In certain embodiments, the pH of the formulation is adjusted with triethanolamine.

It has been observed that greater concentrations of GzmB inhibitor in gel formulations are achieved with increased viscosity enhancer (e.g., Carbopol® 940 or 980) concentration. In certain embodiments, the viscosity enhancer (e.g., Carbopol® 940 or 980) concentration is from about 0.5 to 2 percent by weight of the formulation (e.g. a gel formulation that includes about 10 mg/mL GzmB inhibitor). For a gel formulation that includes, for example, 20 mg/mL GzmB inhibitor, the viscosity enhancer (e.g., Carbopol® 940 or 980) concentration is up to about 5 percent by weight of the formulation.

Representative formulations of the invention include a GzmB inhibitor (0.5 to 15 mg/mL), penetration enhancer (propylene glycol, 15 to 25 percent by weight), viscosity enhancer (Carbopol® 940 or 980, 0.5 to 5 percent by weight), and aqueous acetate buffer at pH 5 (tituated to pH 6.0 with triethanelamine).

In certain embodiments, the formulation can further include one or more preservatives. A suitable preservative includes benzoic acid, EDTA, benzalkonium chloride, parabens, and the like.

A representative paraben includes methyl paraben and propyl paraben (e.g., methyl paraben at about 0.2 and propyl paraben at about 0.02 percent by weight based on the total weight of the formulation).

In one embodiment, the formulation for topical administration is a gel that includes the GzmB inhibitor (e.g., Compound A, Compound 20 or Serpin A3n) at a concentration of 0.35% w/v in a vehicle containing propylene glycol (20% w/w), Carbopol® 940 or 980 (0.5% w/v), methyl paraben (0.2% w/w), propyl paraben (0.02% w/w) and acetate buffer pH 5 (QS), adjusted to the final formulation pH of 5-6 with triethylamine.

In another embodiment, the formulation for topical administration is a gel that includes the GzmB inhibitor (e.g., Compound A, Compound 20 or Serpin A3n) at a concentration of 10 mg/mL in a vehicle containing propylene glycol (20% w/w), Carbopol® 940 or 980 (0.5 to 2.0% w/v), methyl paraben (0.2% w/w), propyl paraben (0.02% w/w), and acetate buffer pH 5 (QS), adjusted to the final formulation pH of 6 with triethanolamine.

In one embodiment, the formulation is an injectable formulation that includes a GzmB inhibitor at a concentration of 0.25 to 25 mg/mL in a pharmaceutically acceptable injection vehicle (e.g., PBS).

The formulations of the invention can further include one or more carriers acceptable for the mode of administration of the preparation, be it by topical administration, lavage, epidermal administration, sub-epidermal administration, intra-epidermal administration, dermal administration, subdermal administration, transdermal administration, subcutaneous administration, subcorneal administration, injection, or any other mode suitable for the selected treatment. Topical administration includes administration to external body surfaces (e.g., skin) as well as to internal body surfaces (e.g., mucus membranes). Suitable carriers are those known in the art for use in such modes of administration.

Suitable compositions can be formulated by means known in the art and their mode of administration and dose determined by a person of skill in the art. For example, A GzmB inhibitor, such as, for example, Compound A, can be dissolved in sterile water or saline or a pharmaceutically acceptable vehicle used for administration of non-water soluble compounds. Many suitable formulations are known including ointments, pastes, gels, hydrogels, foams, creams, powders, lotions, oils, semi-solids, soaps, medicated soaps, shampoos, medicated shampoos, sprays, films, or solutions which can be used topically or locally to administer a compound.

In other embodiments suitable compositions can be pharmaceutical compositions comprising granules, powders, tablets, coated tablets, (micro)capsules, suppositories, syrups, emulsions, suspensions, or preparations with protracted release of the compositions, in whose preparation excipients and additives and/or auxiliaries such as disintegrants, binders, coating agents, swelling agents, lubricants, flavorings, sweeteners or solubilizers are customarily used. The pharmaceutical compositions are suitable for use in a variety of drug delivery systems. For a brief review of methods for drug delivery, see Langer, Science 249, 1527-1533 (1990) and Langer and Tirrell, Nature 428, 487-492 (2004). In addition, the compositions described herein can be formulated as a depot preparation, time-release, delayed release or sustained release delivery system.

The mode of administration can be any medically acceptable mode including oral administration, sublingual administration, intranasal administration, intratracheal administration, inhalation, ocular administration, topical administration as described above, transdermal administration, intradermal administration, intra-epidermal administration, sub-epidermal administration, subcutaneous administration, subcorneal administration, intravenous administration, intramuscular administration, intraperitoneal administration, intrasternal, administration, or via transmucosal administration. In addition, modes of administration can be via an extracorporeal device and/or tissue-penetrating electro-magnetic device.

The particular mode selected will depend upon the particular compound selected, the desired results, the particular condition being treated and the dosage required for therapeutic efficacy. The methods described herein, generally speaking, can be practiced using any mode of administration that is medically acceptable, for example, any mode that produces effective levels of response alteration without causing clinically unacceptable adverse effects.

The compositions can be provided in different vessels, vehicles or formulations depending upon the disorder and mode of administration. For example, for oral application, the compositions can be administered as sublingual tablets, gums, mouth washes, toothpaste, candy, gels, films, and the like; for topical application, as lotions, ointments, gels, creams, sprays, tissues, swabs, wipes, and the like.

The compositions can be administered by injection, e.g., by bolus injection or continuous infusion, via intravenous, subcutaneous, subcorneal, intramuscular, intraperitoneal, intrasternal, intra-epidermal, or sub-epidermal routes. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. For oral administration, the compositions can be formulated readily by combining the compositions with pharmaceutically acceptable carriers well known in the art, e.g., as a sublingual tablet, a liquid formulation, or an oral gel.

For administration by inhalation, the compositions can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of, e.g., gelatin, for use in an inhaler or insufflator can be formulated containing a powder mix of the compositions and a suitable powder base such as lactose or starch. Medical devices for the inhalation of therapeutics are known in the art. In some embodiments the medical device is an inhaler. In other embodiments the medical device is a metered dose inhaler.

Many techniques known to one of skill in the art for the preparation and pharmaceutical formulations are described in Remington: the Science & Practice of Pharmacy by Alfonso Gennaro, 20th ed., Williams & Wilkins, (2000).

The formulations can further include an excipient, a polyalkylene glycol such as polyethylene glycol, an oil of vegetable origin, or a hydrogenated naphthalene. The excipient can be biocompatible, and can include for example, a biodegradable lactide polymer, a lactide/glycolide copolymer, or a polyoxyethylene-polyoxypropylene copolymer.

The formulations of the invention or for use in certain methods disclosed herein can be administered in combination with one or more other therapeutic agents as appropriate. A GzmB inhibitor and pharmaceutical compositions thereof, such as for example, Compound A, in accordance with certain embodiments of the invention described herein or for use in certain methods disclosed herein can be administered by means of a medical device or appliance such as an implant or wound dressing. Also, implants can be devised that are intended to contain and release such compounds or compositions. An example would be an implant made of a polymeric material adapted to release the compound over a period of time.

In certain embodiments, the formulations of the invention “comprise” the described components and can include other components. In other embodiments, the formulations of the invention “consist essentially of” the described components and may include other components that do not materially affect the characteristic properties of the formulation. In other embodiments, the formulations of the invention “consist of” the described components and do not include other components.

In another aspect, the invention provides methods for treating and/or ameliorating symptoms of blisters or peeling skin, healing a blistered and/or peeling skin, reducing or preventing blistering and/or peeling of the skin, blistering and/or peeling of the skin by subcorneal, intra-epidermal, sub-epidermal, subcutaneous, and/or systemic delivery of a GzmB inhibitor.

In certain embodiments, the methods comprise administering a therapeutically effective amount of a GzmB inhibitor or a formulation that includes a GzmB inhibitor to a subject in need thereof. Representative routes of administration include generally topical administration, oral administration, and administration by injection. More specific routes of administration are recited herein.

A “therapeutically effective amount” refers to an amount effective, at dosages and for periods of time necessary, to achieve the desired therapeutic result, such as a reduction in the blistering and/or peeling of skin, a reduction in the blistering and/or peeling of skin, or a reduced level of GzmB activity. A therapeutically effective amount of a compound can vary according to factors such as the disease state, age, sex, and weight of the subject, and the ability of the compound to elicit a desired response in the subject. Dosage regimens can be adjusted to provide the optimum therapeutic response. A therapeutically effective amount is also one in which any toxic or detrimental effects of the GzmB inhibitor are outweighed by the therapeutically beneficial effects.

It is to be noted that dosage values can vary with the severity of the condition to be alleviated. For any particular subject, specific dosage regimens can be adjusted over time according to the individual need and the professional judgment of the person administering or supervising the administration of the compositions. Dosage ranges set forth herein are exemplary only and do not limit the dosage ranges that can be selected by a medical practitioner. The amount of active compound in the composition can vary according to factors such as the disease state, age, sex, and weight of the subject. Dosage regimens can be adjusted to provide the optimum therapeutic response. For example, a single bolus can be administered, several divided doses can be administered over time or the dose can be proportionally reduced or increased as indicated by the exigencies of the therapeutic situation.

In the methods, the administration of GzmB inhibitor can be a local administration (e.g., administration to the site), subcutaneous, intradermal, subcorneal, intra-epidermal, sub-epidermal, and/or a topical administration to a site (e.g., a blister and/or an area of peeling skin). In addition, the GzmB inhibitor can be administered systemically by, for example, orally, intraperitoneally, or intravenously.

The term “subject” or “patient” is intended to include mammalian organisms. Examples of subjects or patients include humans and non-human mammals, e.g., nonhuman primates, dogs, cows, horses, pigs, sheep, goats, cats, mice, rabbits, rats, and transgenic non-human animals. In specific embodiments of the invention, the subject is a human.

The term “administering” includes any method of delivery of GzmB inhibitor or a pharmaceutical composition comprising GzmB inhibitor into a subject's system or to a particular region in or on a subject.

As used herein, the term “applying” refers to administration of the GzmB inhibitor that includes spreading, covering (at least in part), or a layering on of the compound.

As used herein, the terms “treating” or “treatment” refers to a beneficial or desired result including, but not limited to, alleviation or amelioration of one or more symptoms, diminishing the extent of a disorder, stabilized (i.e., not worsening) state of a disorder, amelioration or palliation of the disorder, whether detectable or undetectable. “Treatment” can also mean prolonging survival as compared to expected survival in the absence of treatment.

The following examples are provided for the purpose of illustrating, not limiting, the invention.

### EXAMPLES

### General Methods

Representative compounds of the invention were prepared according to Methods A to C as described and illustrated in FIGS. 8-20 of WO 2017/132771 (incorporated herein by reference in its entirety). The complete methods for preparation of the compounds are found in WO 2017/132771.

It will be appreciated that in the following general methods and preparation of synthetic intermediates, reagent levels and relative amounts or reagents/intermediates can be changed to suit particular compounds to be synthesized, up or down by up to 50% without significant change in expected results.

### Example 1

Separation of the Epidermis from the Dermis by GzmB

In this example, we demonstrate that the epidermis will separate from the dermis in full thickness skin following treatment with GzmB. Abdominal full thickness skin was obtained from elective plastic surgery and used immediately after excision and transport. A small piece of skin (0.2 by 0.4 mm) was immediately fixed in 10% formalin (native skin) while other pieces were placed in 300 μL of phosphate buffer saline (PBS) with or without 200 nM GzmB. Skin samples were then incubated in a water bath at 37° C. for 24 hours. Following incubation the skin was fixed in 10% formaline, paraffin embedded, sectioned (5 μm) and stained with Hematoxylin and Eosin (H&E) using standard methods. FIG. 1 shows the addition of GzmB (200 nM) to fresh human skin leads to separation of the DEJ. Normal skin and vehicle-treated skin are shown in the first two panels. The arrow indicates separation of the epidermis from the dermis in GmzB-treated skin.

In another experiment small pieces of full thickness skin (0.2 by 0.4 mm) were placed in 300 μL of PBS with or without GzmB. Samples were then incubated in a water bath at 37° C. for 24 hours. Following incubation the skin was fixed in 10% formalin, paraffin embedded, sectioned (5 μm) and stained with H&E using standard methods. FIG. 2 shows the addition of GzmB (100 nM) to fresh human skin and separation of the DEJ. Normal skin and vehicle-treated skin are shown in first two panels. Arrow indicates separation of the epidermis from the dermis in GzmB-treated skin.

### Example 2

GzmB Cleavage by Collagen VII and Inhibition Using a GzmB Inhibitor

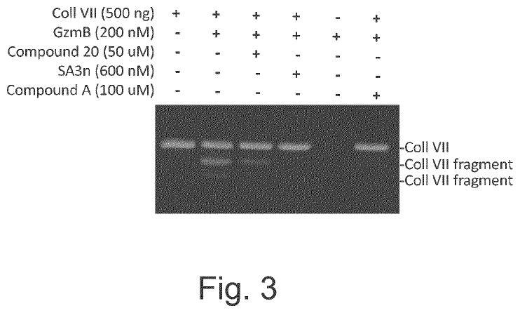

A GzmB cleavage assay was performed in 40 μL of PBS. Briefly, collagen VII (500 ng) was incubated with 200 mM of GzmB for 24 hours at 37° C. For inhibition, GzmB was incubated with Compound 20, Serpin A3N and Compound A for 1 hour at 37° C. prior to the addition of collagen VII. After 24 hours all samples were loaded onto a 10% SDS-PAGE, blotted on PVDF membrane and incubated with a primary antibody specific for collagen VII (rabbit anti-collagen VII, Abcam plc) overnight at 4° C., and then with a secondary labeled anti-rabbit antibody for 1 hour at room temperature. FIG. 3 shows GzmB cleaves collagen VII and that the addition of GzmB inhibitors (SA3N, Compound 20, Compound A) prevented GzmB-mediated collagen VII cleavage.

The cleavage sites within collagen VII for GzmB were determined using TAILS analysing using methods previously described by Kleifeld et al. (Nat. Biotechnol. 28:281-288. doi: 10.1038/nbt.1611, 2010). Briefly, collagen VII was incubated with 200 nM of GzmB for 24 hours at 37° C. N-termini were differentially labeled, denatured, and blocked. Samples were then incubated with trypsin to generate tryptic peptides and then the amine-reactive polymer HPG-ALD to negatively enrich labeled peptides and identify GzmB-cleavage sites.

### Example 3

Identifying Collagen VII at the DEJ in Full Thickness Skin

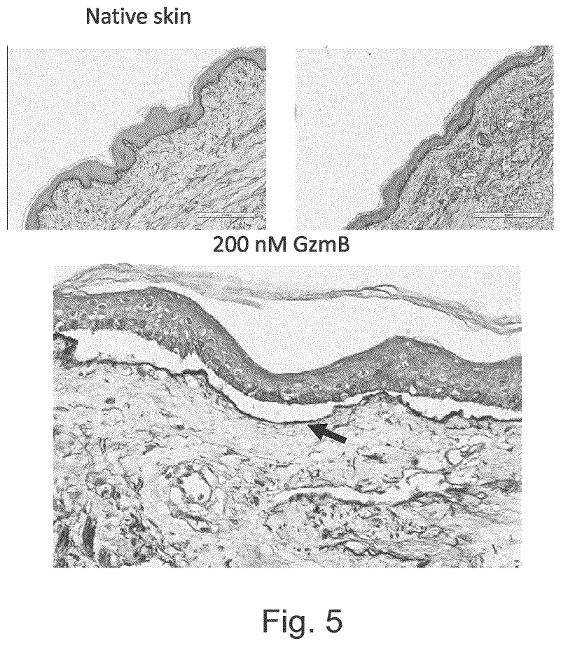

A GzmB skin cleavage assay and paraffin embedding was performed as described for FIGS. 1 and 2. Subsequently, 5 μm sections were deparaffinized and subjected to enzymatic antigen retrieval with trypsin for 15 min using the Carenzyme I: Trypsin Kit (BioCare Medical). Slides were then blocked with 10% goat serum in Tris Buffered Saline (TBS) for 1 hour prior to incubation with rabbit anti-collagen VII antibody (Abcam plc) overnight at 4° C. All slides were then incubated with secondary biotinylated antibody and DAB staining was performed following the manufacturer's instructions. FIG. 5 illustrates immunostaining of collagen VII. Arrow indicates collagen VII staining.

### Example 4

GzmB Cleavage of α6β4 and Inhibition Using a GzmB Inhibitor

In this example it was demonstrated that GzmB can cleave the integrin α6β4 which is a key protein involved in anchoring the extacellular matrix. In addition, changes to α6β4 integrin protein are observed in skin conditions where blistering and skin peeling are observed.

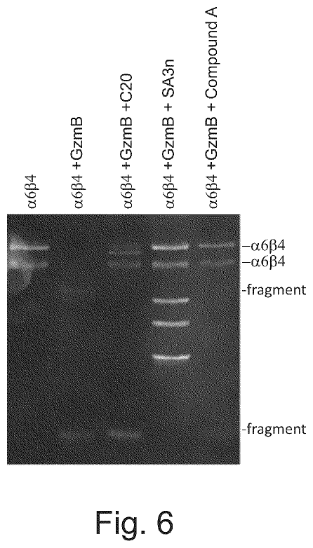

A GzmB cleavage assay was performed in 40 μL of PBS. Briefly, α6β4 (500 ng) was incubated with 200 nM of GzmB for 24 hours at 37° C. For inhibition, GzmB was incubated with Compound 20, Serpin A3N and Compound A for 1 hour at 37° C. prior to the addition of α6β4 integrin. After 24 hours all samples were loaded onto a 10% polyacrylamide gel and separated by electrophoresis for 1.5 hour. Bands were then visualized by staining with a coomassie stain. (FIG. 6).

### Example 5

In this example, a biochemical cleavage assay of the intercellular junction proteins ZO-1, JAM-A, E-cadherin (E-cad), and the desmosomes, Dsg-1 and Dsg-3 was carried out.

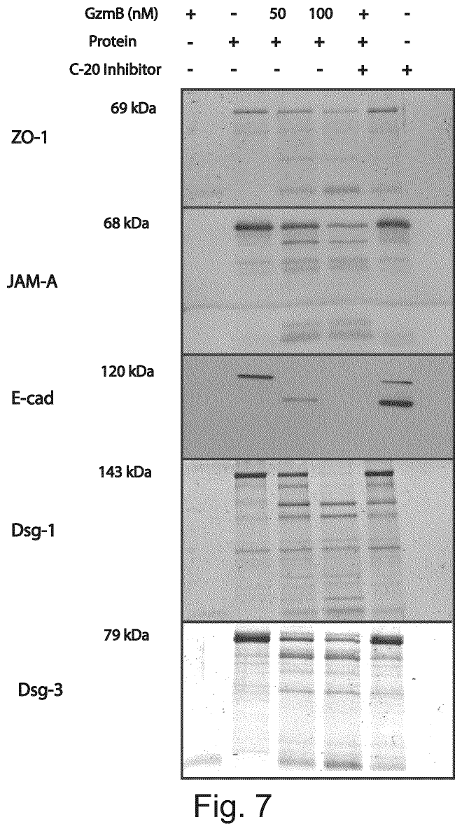

The substrates were incubated with GzmB for 2 hours prior to being separated via SDS-PAGE to identify cleavage fragments. Protein was run on SDS PAGE gel and analyzed by Western blotting (JAM-A, E-cad) or by Coomassie staining (ZO-1, Dsg-1, Dsg-3). All five proteins showed a reduction of whole protein and an increase in fragmentation when incubated with GzmB versus protein alone or GzmB+Compound 20 (C20) inhibitor (FIG. 7). E-cadherin and Dsg-1 showed an almost complete loss of whole protein with 100 nM GzmB, whereas JAM-A, ZO-1, and Dsg-3 showed less cleavage as whole protein was still detectable after GzmB treatment.

### Example 6

Inhibition of Blistering Following the Administration of a GzmB Inhibitor

In this example, a diabetic mouse model was used to show that blistering can be prevented by pretreating the skin with a GzmB inhibitor prior to administration of an event that typically causes skin blistering or after the event, but prior to blister formation. In this example mice were exposed to burn injury by heating a steal rod for 6 seconds to 100° C. to induce blistering.

There were two separate experiments run. Experiment 1 was a 30-day 3-arm study. Arm 1 consisted of treating the mouse daily with Compound A (3.6 mg/mL) in PBS by subcutaneous injection. In arm 2, Compound A (3.6 mg/mL) was administered to the mouse by daily topical application in the gel. In arm 3, the mouse was administered daily by subcutaneous injection saline as a control.

Experiment 2 was a 25-day 2-arm study. Arm 1 consisted of administering daily, through topical application, Compound A (3.6 mg/mL) in the gel. Arm 2 consisted of administering daily the vehicle gel through topical application as a control.

FIG. 8A shows separation of the epidermis from the dermis forming a blister in saline-treated skin. Blistering is prevented in the Compound A-treated skin. FIG. 8B demonstrates that Compound A in PBS (3.6 mg/mL) and in a gel (3.6 mg/mL) were found to reduce blistering as compared to saline-only treatment. FIG. 8C shows that Compound A in gel reduced blistering as compared to the gel when used alone. FIGS. 8A through 8C therefore provide evidence a GzmB inhibitor (Compound A) can prevent blistering.

GzmB Inhibitor Formulation

The GzmB inhibitor composition including Compound A was formulated as a gel at 3.6 mg/mL Compound A based on the volume of the gel.

Procedure for Making the Base Vehicle

The base vehicle included 20% propylene glycol (PG), 0.2% methyl paraben, 0.02% propyl paraben, in acetate buffer (10 mM, pH 5). The base vehicle was prepared by mixing 20% of PG with acetate buffer (10 mM, pH 5). An excess amount of methyl paraben and propyl paraben (0.2% methyl paraben/0.02% propyl paraben) was added to the solution, stirred overnight (>8 hours) at room temperature. The pH of the solution was adjusted to pH 5 with 1M HCl. The final mixture was filter via 0.45 μm filter.

Procedure for Making the Gel Formulations:

For the pre-clinical lab scale (non-sterile), a 13 mg/mL formulation was prepared by adding Compound A into the vehicle (20% PG, 0.2% methyl paraben, 0.02% propyl paraben, acetate buffer (10 mM, pH 5), prepared as described above) and sonicated for 1 hour. 1% Carbopol 940 NF was added to the formulation and the mixture was stirred for 24 hours at room temperature in order to fully hydrate the Carbopol. After 24 hours, the pH was adjusted to pH 6.0±0.2 with triethanolamine. The final formulation is a colorless transparent gel. The formulation is physically and chemically stable with over 90% Compound A recovery by UPLC-UV up to 1 month at refrigeration storage conditions (2-8° C.).

The formulation can be sterilized. For example, the hydrated Carbopol mixture can be sterilized via autoclave process and the Compound A solution can be filtered via 0.22 μm filtration, the combination process can be performed in a sterilized environment.

### Example 7

Immunohistochemistry and Histology on Diseased Skin

Paraffin-embedded skin samples from patients affected by bullous pemphigoid, dermatitis herpetiformis, and epidermolysis bullosa acquisita (EBA) were sectioned (5 μm) for immunohistochemical analysis of GzmB (ABCAM, Toronto, ON, Canada) and collagen VII (ABCAM, Toronto, ON Canada) using 3,3′-Diaminobenzidine for visualization, as well as α6 (ABCAM) and β4 (ABCAM) integrins, and collagen XVII (generous gift from Dr. Claus-Werner Franzke, University Medical Centre Freiburg, Freiburg, Germany) visualized through Novared®. Healthy skin obtained from patients undergoing elective abdominoplasty was used as a control. In order to observe cellular infiltrate and tissue architecture, Hematoxylin and Eosin (H&E) staining was also performed using established methods. GzmB-producing immune cells were detected by de-staining H&E slides and probing the same section for GzmB. All slides were scanned using a Aperio CS2 slide scanner (Leica, Concord, ON).

Ex vivo sections of human skin blisters from patients with different pemphigus subtypes were also examined to establish whether GzmB was present in the epidermis in or around the lesion. Formalin fixed, paraffin embedded pemphigus blister tissues were analyzed by hematoxylin & eosin (H&E) staining and immunostaining for GzmB. Compared with healthy skin, there was a diffuse GzmB staining within the epidermis and sites of acantholysis are noted in pemphigus herpetiformis, pemphigus foliaceus, and pemphigus vegetans. The largest increase in levels of GzmB was seen within the blister and blister fluid, especially in IgA pemphigus, Hailey-Hailey disease, and limited oral pemphigus vulgaris. While pemphigus vulgaris and paraneoplastic pemphigus showed lower levels of GzmB however, there was still evidence of GzmB accumulation within the dermis.

DEJ Proteins Cleavage Assay

The recombinant human integrin α6/β4 (α6 a.a. 24-878, β4 a.a. 28-710, R&D Systems, Minneapolis, Minn.), collagen VII (199-482 a.a., MyBiosource, San Diego, Calif.), collagen XVII (a.a. 490-1497, generous gift from Dr. Claus-Werner Franzke), and collagen I (Novus Biologicals, Littleton, Colo.) were incubated for 24 hours at 37° C. in 200 nM purified human GzmB (EmeraldBio, Bainbridge Island, Wash.). For inhibition studies, prior to the addition of substrates to the reaction, GzmB was incubated in the presence of 600 nm serpin A3N (generous gift from Dr. Chris R. Bleackley, University of Alberta, Edmonton, AB, Canada) or 100 NM of the small molecule inhibitor Compound 20 (courtesy of the Centre for Drug Research and Development, Vancouver, BC) for 1 hour at 37° C. After the 24 hours incubation, proteins were denatured, separated on a 4-20% (for integrin α6/β4), 10% (for collagen VII), 8-10% (for collagen XVII), or 7.5% (for collagen I) SDS-polyacrylamide gel. Cleavage was detected by Western Blot using anti-human integrin α6 sub-unit (ABCAM), anti-human integrin β4 sub-unit (R&D Systems, Minneapolis, Minn.), anti collagen VII (ABCAM), and anti-human collagen XVII (generous gift from Dr. Claus-Werner Franzke) and imaged using a Li-Cor Odissey® FC (Li-Cor, Lincoln, Nebr.). Coomassie staining was used for the assessment of collagen I cleavage following manufacturer instructions.

LC-MS/MS Analysis

GzmB cleavage sites were identified by ATOMS as described earlier (Doucet and Overall, Meth. Enzymol. 501:275-293, 2011; Doucet and Overall, Mol. Cell Proteomics 10:M110.003533, 2011). Briefly, GzmB-digested or control substrates were denatured, cysteines reduced with dithiothreitol and alkylated with iodoacetamide. Primary amine groups were dimethylated with formaldehyde and sodium cyanoborohydride before acetone precipitation. Pellets were redissolved in digestion buffer with 1 μg/mL MS-grade trypsin (Thermo Fisher Scientific, Waltham, Mass.) and desalted using StageTips™ (Rappsilber et al., Nat. Protoc. 2:1896-1906, 2007). Samples were analyzed on an Impact II™ Q-TOF (Bruker, Billerica, Mass.) on ReproSilPur™ 120 C18-AQ 1.9 μm particles (Dr. Maisch) columns and resolved by a gradient of acetonitrile (0.1% v/v formic acid) in water delivered by an easy-n LC™ system (Thermo Fisher Scientific) preceding data-dependent precursor selection of the top 18 peaks. Spectra were extracted using DataAnalysis 4.3 (Bruker) and searched against a Uniprot human proteome database (downloaded 2016-02-24; 70,472 sequences) using semi-specific ArgC as enzyme specificity, carbamidomethylation (C) and dimethylation (K) as fixed modifications, and deamidation (N,Q), dimethylation (N-term), pyroglutamation (Q) and oxidation (M) as variable modifications. Potential GzmB cleavage sites were defined as semi-specific, N-terminally dimethylated peptides with an acidic residue N-terminal to the identified sequence identified at a peptide expect value <0.01.

Skin Cleavage Assay

Fresh healthy human skin obtained from patients undergoing elective plastic surgery was transported to the lab and cut into z 1 mm×4 mm strips. The adipose tissue layer was removed to obtain a strip of dermis and epidermis. Skin strips were then incubated at 37° C. for 12 hour in 300 μL of either PBS, 200 nM GzmB, or 200 nM GzmB previously inactivated through 1 hour incubation at 37° C. with 100 μM Compound 20. Following incubation, samples were fixed in 10% buffered formalin overnight, paraffin embedded and sectioned for H&E staining using established methods. Outermost sections of the samples were used for staining, as GzmB might not penetrate deep into the tissue.

Results

GzmB Accumulates at the Level of the DEJ in Bullous Pemphigoid, Dermatitis Herpetiformis, and EBA

Immunohistochemistry of patient skin samples from bullous pemphigoid, dermatitis herpetiformis, and EBA indicated that GzmB accumulated at the DEJ.

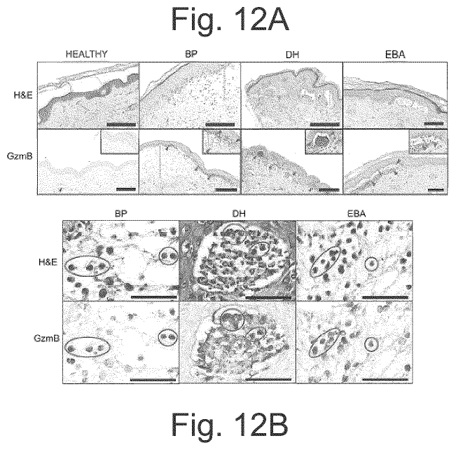

Specifically, H&E staining of bullous pemphigoid revealed sub-epidermal blistering with dense inflammatory infiltrate consisting predominantly of eosinophils and neutrophils (FIGS. 12A and 12B). Intense GzmB staining was observed at the level of the DEJ in most neutrophils but not in eosinophils, both within the blister and immediately below the detached epidermal layer (FIG. 12B). Dermatitis herpetiformis was characterized by pathognomonic sub-epidermal clefts and papillary abscesses, consisting mostly of neutrophils and a few eosinophils, at the tips of dermal papillae (FIGS. 12A and 12B). These papillary abscesses were heavily stained for GzmB, suggesting neutrophil and lymphocyte involvement in its secretion (12B). GzmB presence was predominantly observed in the upper papillary dermis adjacent to the DEJ, but positive cells could also be detected embedded within the epidermal layer. EBA skin displayed epidermal detachment with abundant immune infiltrate, mostly composed of neutrophils and lymphocytes, in the interstitial space between the separated epidermis and the dermis (FIGS. 12A and 12B). Similar to what observed in BP and DH, GzmB was found predominantly in neutrophils (FIG. 11B).

GzmB Cleaves α6 and β4 Integrins In Vitro in Domains Pivotal for their Function

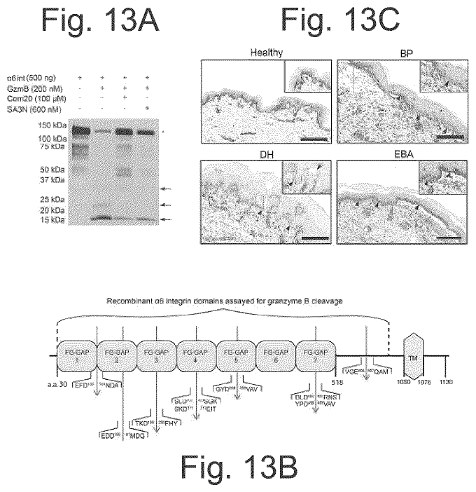

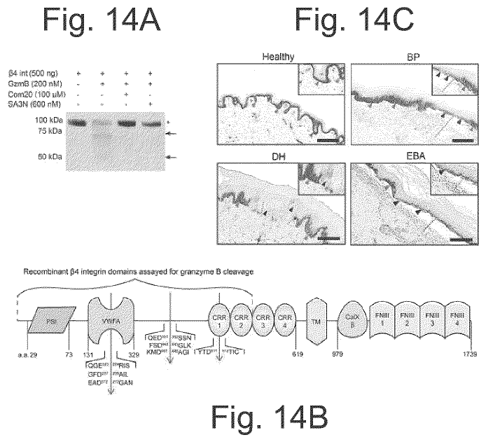

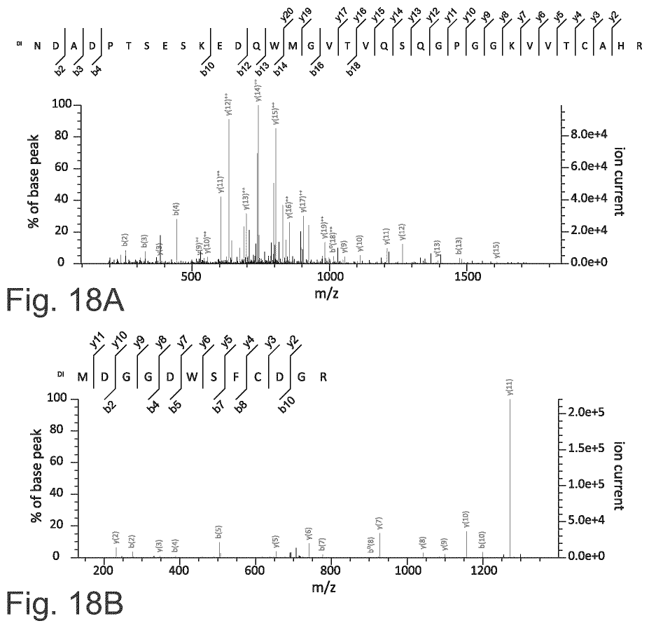

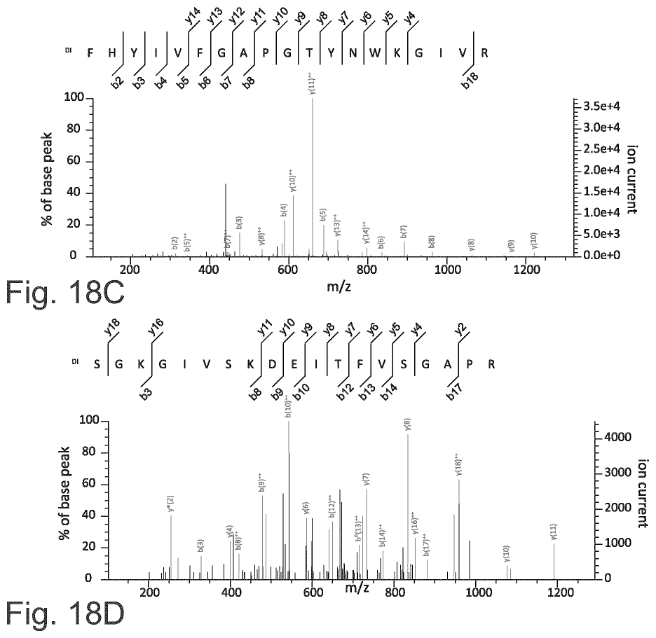

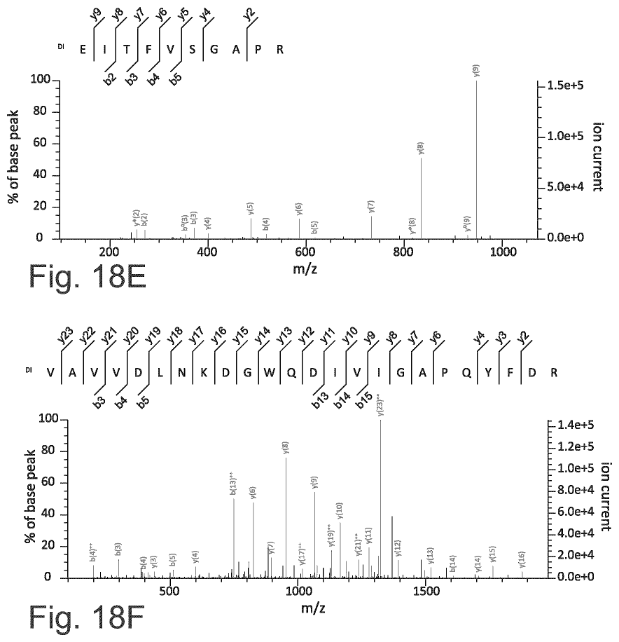

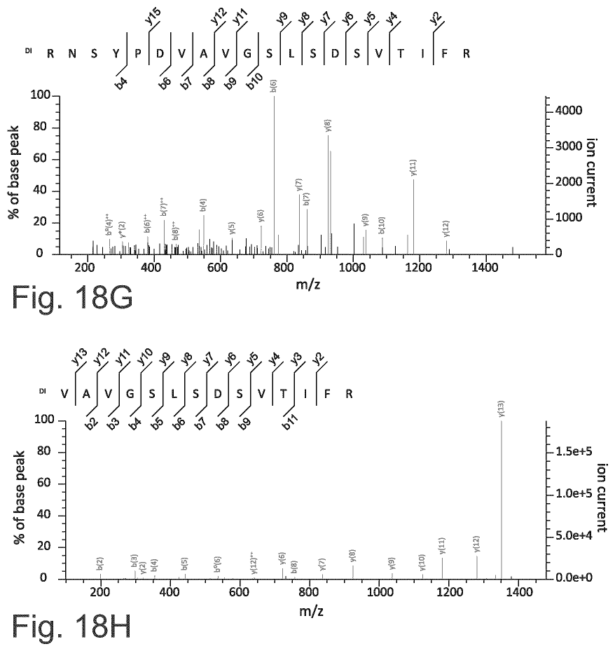

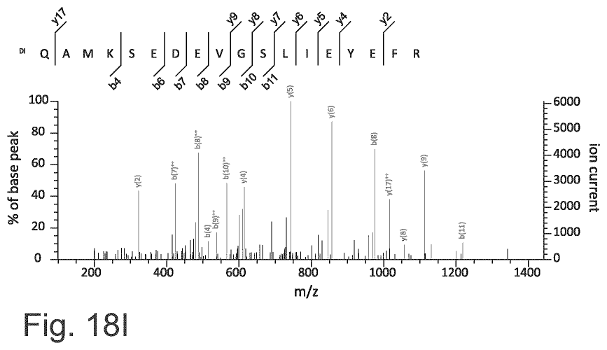

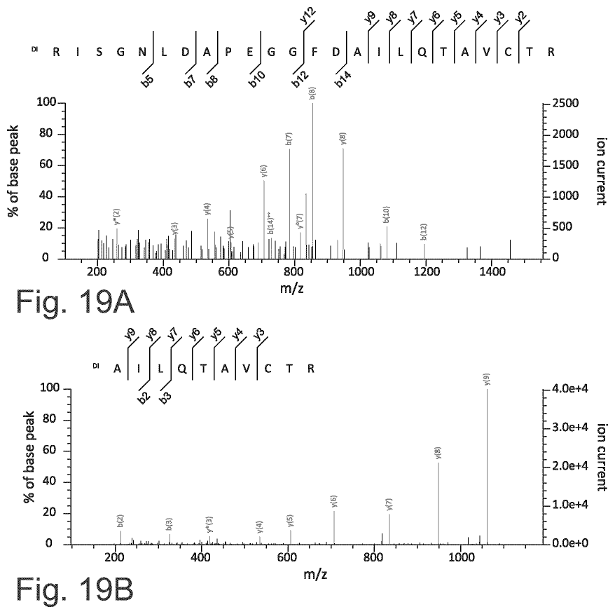

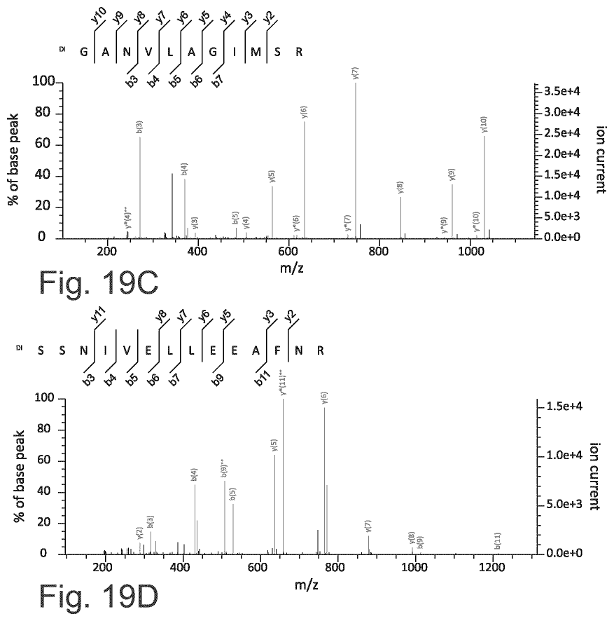

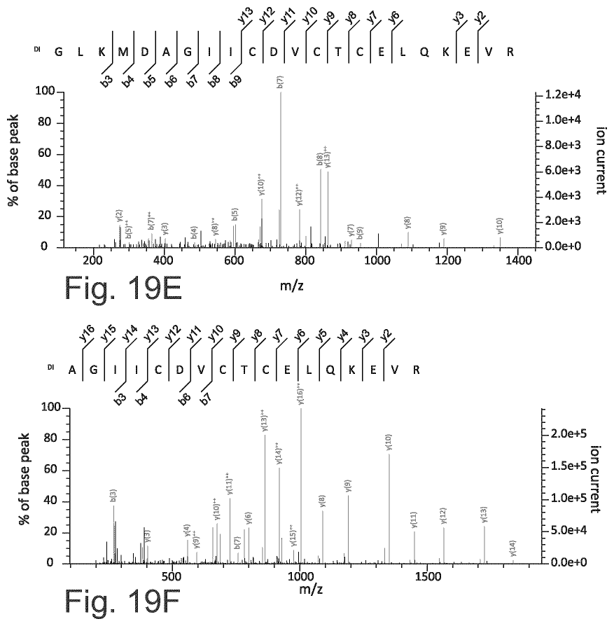

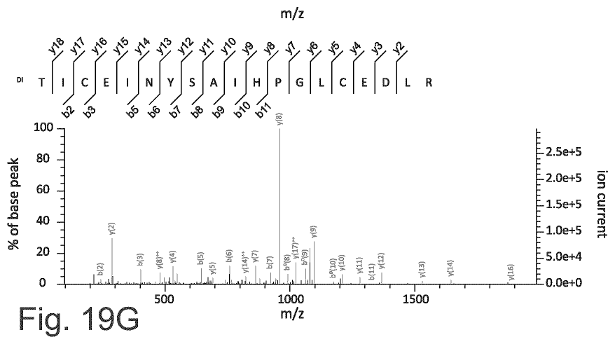

Once the presence of GzmB was ascertained at the level of the DEJ in diseased skin, we hypothesized that GzmB mediates cleavage of α6/β4 integrin, collagen VII, and collagen XVII, which are important components of the basement membrane critical for DEJ function. Both α6 and β4 integrin sub-units were cleaved by GzmB (FIG. 13A and FIG. 14A); full length α6 integrin was detected at 150 kDa with fragments at ˜20, 25, and 37 kDa, whereas cleavage of full length β4 (100 kDa) yielded an evident fragment at ˜65 kDa and a weaker band at 50 kDa. To confirm that cleavage of these DEJ components was indeed mediated by GzmB, compound 20, a GzmB-specific competitive inhibitor, and serpin A3N, an irreversible serine protease inhibitor, were included in the cleavage assay. Both inhibitors prevented the cleavage of α6 and (β4 integrin sub-units at 100 μM and 600 nM respectively (FIG. 13A and FIG. 14A). Furthermore, mass spectrometry by ATOMS was used to identify cleavage sites on these proteins to assess whether GzmB mediated cleavage of α6 and (β4 integrins could impair DEJ function. We focussed on the extracellular domains of α6 and β4 integrins as this protein region is more likely to be exposed to GzmB. α6 integrin was cleaved by GzmB at Asp100, Asp166, Asp199, Asp302, Asp311, Asp358, Asp482, and Asp488 in the FG-GAP repeats 2, 3, 4, 5, and 7 within the extracellular β-propeller domain (FIG. 13B and FIGS. 18A through I), a cleavage site at Glu856 was also detected. Cleavage of the β4 integrin sub-unit fell within the Von Willebrand factor A domain at Glu223, Asp237 and Asp272, as well as within the Cysteine Rich Region 1 at Asp611, and within the linker region between these two domains at Asp351, Asp442 and Asp447 (FIG. 13B and FIGS. 19A through G).

GzmB Cleaves Collagen VII in Ligand Binding Regions

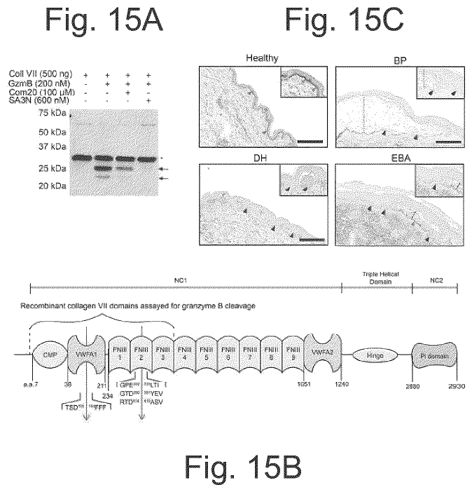

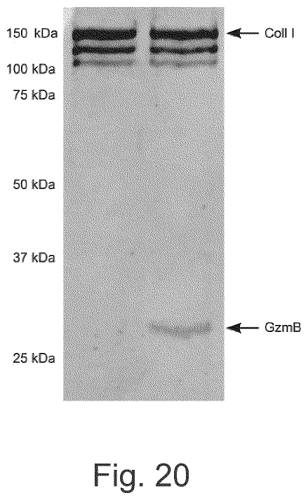

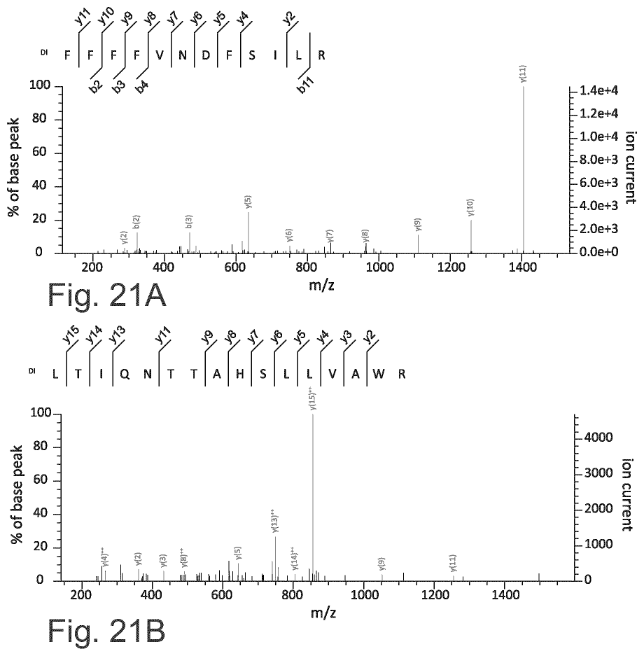

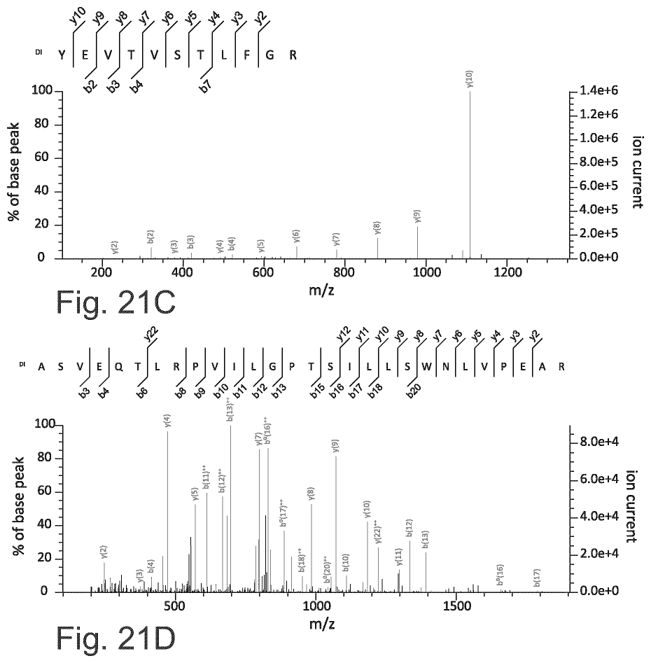

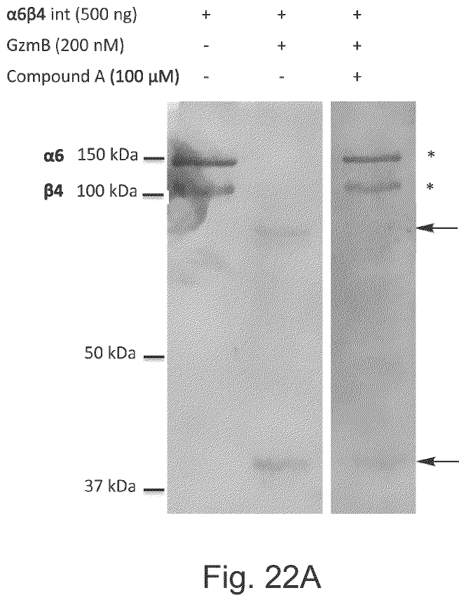

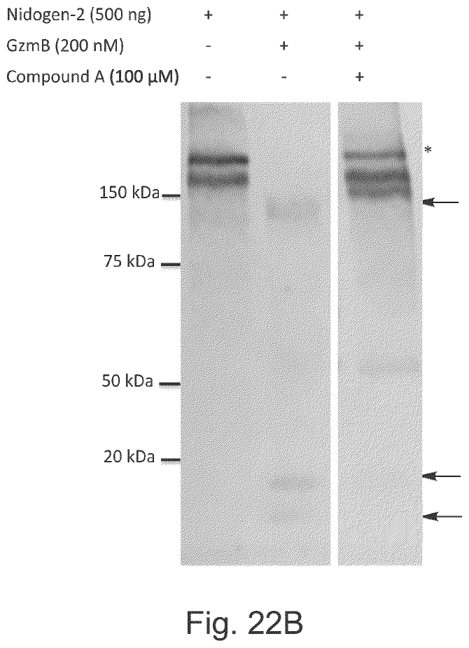

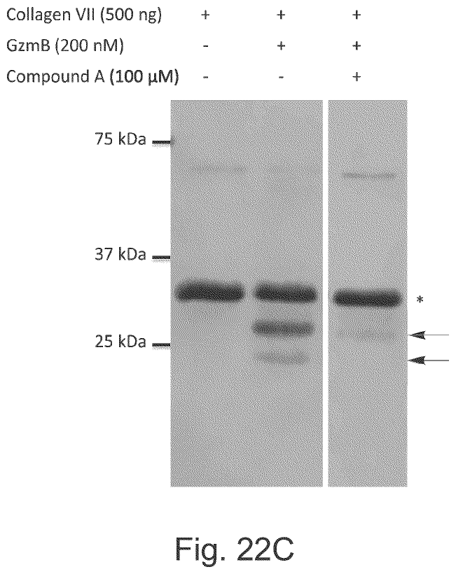

Western Blot was used to assess cleavage of collagen VII domain a.a 199-482 by GzmB. This fragment is part of the non-collagenous region 1 (NC1), which is pivotal for collagen VII interactions with other proteins of the ECM (Chen et al., J. Biol. Chem. 272:14516-14522, 1997; Chen et al., J. Invest. Dermatol. 112:177-183,1999). Untreated collagen VII fragment was detected at ˜30 kDa, and its cleavage by GzmB yielded bands at ˜20 and 25 kDa (FIG. 15B). On the other hand, collagen I, the most common collagen in the human body, was not cleaved by GzmB (FIG. 20). Inhibition of collagen VII cleavage with serpin A3N prevented the appearance of both ˜20 and 25 kDa bands, whereas Compound 20 inhibition was incomplete and a cleavage band could still be detected at ˜25 kDa (FIG. 15B). As for the location of cleavage, the NC1 fragment of collagen VII we tested was cleaved by GzmB in the Von Willebrand factor A domain at Asp193, in the fibronectin-like domain III-2 at Glu332 and Asp390, and within fibronectin-like domain III-3 at Asp414 (FIG. 15B and FIGS. 21A through 21C).

Collagen XVII is a Substrate for GzmB Cleavage

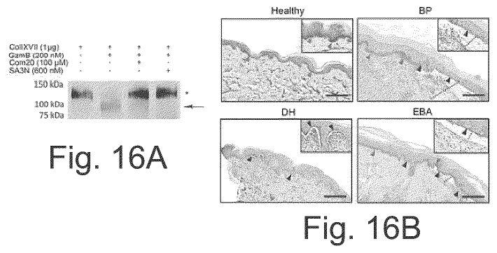

As a crucial component of the hemidesmosomes, collagen XVII plays a critical role in bridging the intracellular and the extracellular structural elements involved in epidermal adhesion (Franzke et al., J. Biol. Chem. 280:4005-4008, 2005). Treatment of collagen XVII NC16 ectodomain (a.a 490-1497) with GzmB resulted in cleavage of this region, and in the appearance of a cleavage band at ˜100 kDa (full length protein˜130 kDa). Pre-incubation of GzmB with both Compound 20 and serpin A3N prevented NC16 cleavage (FIG. 16A). ATOMS was attempted on collagen XVII using 1 or 2 μg of protein. GzmB-digested and undigested forms of collagen XVII were compared through heavy (¹³CD₂O formaldehyde) and light (¹²CH₂O formaldehyde) dimethylated tags respectively.

α6/β4 Integrin, Collagen VII, and Collagen XVII Lining at the DEJ are Disrupted in Diseased Skin

Following Western Blot and ATOMS analyses indicating that both α6 and β4 integrin sub-units and collagen VII are substrates for GzmB, we sought to assess their integrity in bullous pemphigoid, dermatitis herpetiformis, and EBA skin samples. Staining for α6 integrin in normal skin was mainly localized perivascularly in the dermis and as a continuous line at the DEJ. When diseased skin samples were investigated, the pattern of α6 integrin localization was similar for all conditions, exhibiting scattered staining throughout the entire sections, which could be indicative of protein fragmentation (FIG. 13C). At the DEJ, staining was absent, weak, or disorganized both in areas of epidermal detachment and in sections where the epidermis was still attached to the dermis (FIG. 13C). As for integrin β4, a strong well localized staining delineated the DEJ in healthy skin, as well as in bullous pemphigoid and dermatitis herpetiformis in areas where the epidermis was anchored to the dermis (FIG. 14C). However, integrin β4 staining was completely absent or faint in areas of epidermal separation in all conditions studied (FIG. 14C).

Mooney et al. (J. Cutan. Pathol. 18:417-422, 1991) have previously shown fragmented collagen VII in the DEJ of patients with discoid lupus erythematosus. While normal skin displayed a continuous, strong staining for collagen VII lining the interface between epidermis and dermis (FIG. 15C), in bullous pemphigoid and dermatitis herpetiformis collagen VII staining was weak or absent. Weak staining in both diseases was localized on the dermal side of a blister or papillary abscess, consistent with a separation of the fibrillar zone of the DEJ from the lamina densa above it due to cleavage of collagen VII in the NC1 (FIG. 15C). In dermatitis herpetiformis, collagen VII staining presented a peculiar pattern, with short stained sections perpendicular to the epidermis rather than parallel to it. Collagen VII staining of EBA samples showed an intermittent pattern, with DEJ segments presenting weak staining alternated by areas with a stronger staining (FIG. 15C).

Finally, collagen XVII staining pattern for all conditions was similar to what was observed for the other substrates. A clear, uninterrupted line of staining was detected in healthy skin, whereas in diseased skin weak staining was observed in areas of reduced DEJ integrity and was mostly absent in the sections of skin where the epidermis had detached (FIG. 16B).

Incubation with GzmB Results in Epidermal Separation in Healthy Human Skin and is Inhibited by Compound 20

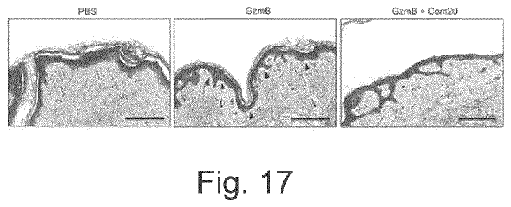

As GzmB is abundant at the DEJ of bullous pemphigoid, dermatitis herpetiformis, and EBA, and capable of cleaving key junctional proteins, the direct impact of GzmB proteolysis on DEJ integrity in freshly-isolated human skin was assessed. A ˜1 mm×4 mm strip of healthy skin comprising epidermis and dermis was immersed and incubated for 12 hours at 37° C. in a 200 nM solution of GzmB. Upon incubation, H&E staining revealed the appearance of clefts between the epidermis and the dermis in the GzmB-treated sample that were mostly absent in the PBS control (FIG. 17). Inactivation of GzmB with the GzmB-specific inhibitor Compound 20 prevented the formation of DEJ clefts, revealing a tissue morphology similar to PBS control (FIG. 17).

### DISCUSSION

It is now widely acknowledged in the literature that extracellular GzmB exerts a pathogenic, perforin-independent, role in conditions associated with dysregulated and/or chronic inflammation and impaired tissue repair due to ECM cleavage (Boivin et al., PloS ONE 7:e33163, 2012; Hiebert et al., Cell Death Differ. 20:1404-1414, 2013; Parkinson et al., Aging Cell 14:67-77, 2014; Shen et al., Am. J. Pathol. 186:87-200, 2016). In the present study, Western Blot and ATOMS were used to show for the first time that α6/β4 integrin and collagen VII, which are key components of the DEJ, are cleaved by GzmB in key regions for their anchoring functions. It was also demonstrated that in diseased human skin biopsies of bullous pemphigoid, dermatitis herpetiformis and EBA, sub-epidermal blisters display elevated levels of GzmB at the DEJ accompanied by degradation of the newly discovered GzmB substrates α6/β4 integrin, collagen VII, and collagen XVII. Supporting the pathological relevance of GzmB activity, we reported partial detachment of the epidermis from the dermis in healthy human skin exposed to a physiologically relevant concentration of GzmB, and showed that this separation can be prevented by a GzmB-specific inhibitor. These data suggest a common extracellular role for GzmB in different autoimmune sub-epidermal blistering pathogenesis.

Evidence for the importance of α6/β4 integrin comes from both animal models (van der Neut et al., Nat. Genet. 13:366-369, 1996) and from severe, often lethal, human blistering diseases. Mutations and deficiencies of the integrin complex involving α6/β4, as well as production of anti-α6/β4 integrin auto-antibodies (Leverkus et al., Br. J. Dermatol. 145:998-1004, 2001; Kiss et al., Ann. NY Acad. Sci. 1051:104-110, 2005), contribute to lethal phenotypes characterized by widespread muco-cutaneous blistering. Several studies have identified mutations in the genes coding for α6/β4 integrin (Ruzzi et al., J. Clin. Invest. 99:2826-2831, 1997; Pulkkinen et al., Am. J. Hum. Genet. 63:1376-1387, 1998; Jonkman et al., J. Invest. Dermatol. 119:1275-1281, 2002) or absence at the protein level of one of its sub-units (Niessen et al., J. Cell Sci. 109(Pt 7)1695-1706, 1996), in numerous subtypes of epidermolysis bullosa. In particular, Phillips et al. speculated that in situ proteolytic cleavage of the epitopes by an as yet unidentified protease, might be responsible for the loss of β4 subunit immunoreactivity in patients with junctional epidermolysis bullosa (Phillips et al., Histopathology 24:571-576, 1994). In this study, we show that GzmB cleaves both α6 and β4 integrin sub-units at several sites in their ligand-binding extracellular domains (Tuckwell and Humphries, FEBS Lett. 400:297-303, 1997; Oxvig and Springer, Proc. Nat'l. Acad, Sci. USA 95:4870-4875, 1998; Tsuruta et al., J. Biol. Chem. 278:38707-38714, 2003; Pawar et al., Exp. Cell Res. 313:1080-1089, 2007), possibly compromising the adhesive properties of this molecule. The importance of these extracellular regions is particularly evident for integrin β4 since most of the missense mutations and the amino acid deletions described in lethal junctional epidermolysis bullosa were located in its extracellular domain (Pulkkinen et al., AJPA 152:935-941, 1998; Pulkkinen et al., AJPA 152:157-166, 1998; Pulkkinen et al., Am. J. Hum. Genet. 63:1376-1387, 1998; Nakano et al., Pediatr. Res. 49:618-626, 2001), while missense or splice mutations associated with the nonlethal form were frequently located in the cytoplasmic portion (Kambham et al., Am. J. Kidney Dis. 36:190-196, 2000; Nakano et al., Pediatr. Res. 49:618-626, 2001; Koster et al., J. Cell Sci. 116(Pt 1):387-399, 2003). Interestingly, Nakano et al. identified the lethal mutation p.D131Y/p.G273D, which may abolish important ligand binding sites of integrin β4 as it falls within in a highly conserved region (Nakano et al., Pediatr. Res. 49:618-626, 2001). Since one of the GzmB cleavage sites we have identified is at Asp272, this demonstrates that GzmB-mediated cleavage of α6/β4 integrin could likewise severely affect the adhesive properties of this molecule.

As the main component of the papillary dermis, another protein fundamental for the integrity of the DEJ is collagen VII. Among collagen VII domains, fibronectin-like regions are pivotal for the interaction with several ECM components. Studies with a recombinant version of the NC1 region revealed strong binding affinity of fibronectin-like domains with collagen I, collagen IV, laminin 332 and fibronectin (Chen et al., J. Biol. Chem. 272:14516-14522, 1997; Chen et al., J. Invest. Dermatol. 112:177-183, 1999), which allows anchorage of the papillary dermis to the lamina densa. Mutations of collagen VII or production of auto-antibodies against this region result in severe disruption of the DEJ, causing dystrophic epidermolysis bullosa and EBA respectively (Dang and Murrell, Exp. Dermatol. 17:553-568, 2008; Kim and Kim, J. Eur. Acad. Sermatol. Venereol. 27:1204-1213, 2013). These conditions are characterized by extensive epidermal detachment and formation of blisters. GzmB-mediated cleavage of collagen VII in the fibronectin-like III-2 domain is shown herein, as well as in the von Willebrand factor A domain, and inhibition of this cleavage by the GzmB inhibitors serpin A3N and compound 20 (Willoughby et al., Bioorg. Med. Chem. Lett. 12:2197-2200, 2002). Moreover, GzmB-mediated cleavage of collagen XVII was also observed, another important component of the hemidesmosome, whose interaction with α6/β4 integrin is required for the assembly of protein complexes that anchor basal keratinocytes to the lamina lucida (Koster et al., J. Cell Sci. 116(Pt2):387-399, 2003). Taken together, these observations show that GzmB can disrupt the basal keratinocytes/lamina lucida connection through cleavage of α6/β4 integrin and collagen XVII, and lamina densa/fibrillar zone adhesion through cleavage of collagen VII.

GzmB accumulation at the DEJ is observed in many interface dermatoses, including for example, SJS/TEN and generalized bullous fixed drug eruption (Cho et al., J. Am. Acad. Dermatol. 70:539-548, 2014). However, none have considered an extracellular role for this protease. Rather, GzmB was proposed to contribute to CD8⁺ T-cell- and NK-mediated, keratinocyte and melanocyte death in a perforin-dependent manner. Our results indicate that this hypothesis does not explain the pathologic role for GzmB in blistering. While mostly absent in normal skin, an abundance of GzmB was observed at the DEJ in the autoimmune sub-epidermal blistering conditions bullous pemphigoid and dermatitis herpetiformis, in agreement with previous studies (Hussein et al. J. Clin. Pathol. 60:62-71, 2007; Abreu Velez et al., Our Dermatol. Online 4:627-630, 2013), and shown for the first time an accumulation of this protease at the DEJ in EBA. It has long been hypothesized that anti-DEJ auto-antibody-triggered sub-epidermal blister formation is mediated by proteases secreted by infiltrating inflammatory cells (Jordon et al., J. Invest. Dermatol. 85(Suppl):72s-78s, 1985). Indeed, auto-antibody-mediated immune cell recruitment to the DEJ is a mandatory condition for dermo-epidermal separation as it dictates the localized and concentrated degranulation of proteinases (Sitaru and Zillikens, Exp. Dermatol. 14:861-875, 2005). Confirming this mechanism, auto-antibodies contained in the serum of patients with bullous pemphigoid (Mihai et al., J. Cell Mol. Med. 11:1117-1128, 2007), EBA (Sitaru et al., AJPA 161:301-311, 2002), and pemphigoid gestationis (Herrero-Gonzilez et al. Eur. J. Immunol. 36:1039-1048, 2006) promote leukocyte recruitment to the DEJ resulting in its separation. GzmB can be an important contributor to this DEJ separation in autoimmune conditions as the DEJ substrates now identified herein are degraded proximal to the area in which blistering is occurring in these diseases.

The pathological role of extracellular GzmB in autoimmune diseases might not be limited to the physical disruption of important substrates. Growing evidence suggests that antigenic, GzmB-generated peptide fragments are part of a feed-forward loop that sustains the propagation of several autoimmune diseases (reviewed in Darrah and Rosen, Cell Death Differ. 17:624-632, 2010). Studies suggest that GzmB is instrumental in auto-antigen generation in certain autoimmune conditions (Nagaraju et al., Arthritis Rheum. 44:2376-2386, 2001; Niland et al., J. Immunol. 184:4025-4032, 2010; Darrah et al., J. Proteome Res. 16:355-365, 2017). In this disclosure, GzmB is demonstrated to cleave α6/β4 integrin, collagen XVII, and collagen VII in epitope regions recognized by auto-antibodies present in the sera of patients with certain pemphigoid diseases, bullous pemphigoid, and EBA respectively. In oral pemphigoid, one of the identified auto-epitopes is represented by the peptide a.a. 292-305 of α6 integrin (Rashid et al., J. Immunol. 176:1968-1977, 2006). In vitro, we showed that GzmB cleaves α6 integrin at residues Asp199 and Asp302, thus GzmB can potentially generate this antigenic fragment in vivo, establishing a cycle of sustained immune response and further generation of antigenic fragments.

As mentioned above, GzmB-mediated cleavage of the NC16 region of collagen XVII was also observed. The production of collagen XVII auto-antibodies results in bullous pemphigoid (Zimina et al., J. Invest. Dermatol. 128:2736-2739, 2008; Nishie, J. Dermatol. Sci. 73:179-186, 2014) while mutations in the NC16 ectodomain of this protein have been associated with certain forms of junctional epidermolysis bullosa (McGrath et al., AJPA 148:1787-1796, 1996; Schumann et al., Am. J. Hum. Genet. 60:1344-1353, 1997). Although the specific GzmB cleavage sites in the NC16 region of collagen XVII was not identified through ATOMS, this domain is the immunodominant region in bullous pemphigoid and its recombinant forms are used for detecting specific autoantibodies in approximately 85% of patients affected by this condition (Giudice et al., J. Immunol. 151:5742-5750, 1993; Murakami et al., J. Dermatol. Sci. 13:112-117, 1996).

Finally, several groups have demonstrated that in EBA, T and B cells target identical regions of the NC1 domain of collagen VII (Jones et al., J. Invest. Dermatol. 104:231-235, 1995; Mutller et al., Clin. Immunol. 135:99-107, 2010). In particular, Lapiere et al. incubated different fragments of collagen VII with sera from 19 EBA patients, and observed that 16 sera strongly reacted with the fusion protein composed of the fibronectin-like III domains 1 to 4 (Lapiere et al., J. Clin. Invest. 92:1831-1839, 1993). The NC1 domain of collagen VII also mediates Fc-dependent neutrophil activation and induction of dermo-epidermal separation (Sitaru et al., AJPA 161:301-311, 2002).

In summary, the present study shows for the first time that GzmB extracellular proteolysis directly contributes to subcorneal, intra-epidermal, and sub-epidermal blistering via DEJ impairment in autoimmune blistering skin conditions. Inhibition of GzmB represents a novel therapeutic approach for the treatment and prevention of subcorneal, intra-epidermal and sub-epidermal blistering, including autoimmune blistering.

While a preferred embodiment of the invention has been illustrated and described, it will be appreciated that various changes can be made therein without departing from the spirit and scope of the invention.

The embodiments of the invention in which an exclusive property or privilege is claimed are defined as follows:

1. A composition for treating and/or preventing blistering and/or peeling of a skin, comprising a compound having Formula (I) and a pharmaceutically acceptable carrier, wherein Formula I comprises: 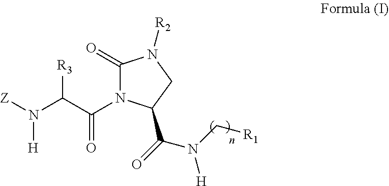
stereoisomers, tautomers, or pharmaceutically acceptable salts thereof, wherein:
R₁ is a heteroaryl group selected from
(a) 1,2,3-triazolyl, and
(b) 1,2,3,4-tetrazolyl;
n is 1 or 2;
R₂ is selected from hydrogen, C₁-C₆ alkyl, and C₃-C₆ cycloalkyl;
R₃ is selected from
(a) hydrogen,
(b) C₁-C₄ alkyl optionally substituted with a carboxylic acid, carboxylate, or carboxylate C₁-C₈ ester group (—CO₂H, —CO₂, —C(═O)OC₁-C₈), an amide optionally substituted with an alkylheteroaryl group, or a heteroaryl group;
Z is an acyl group selected from the group 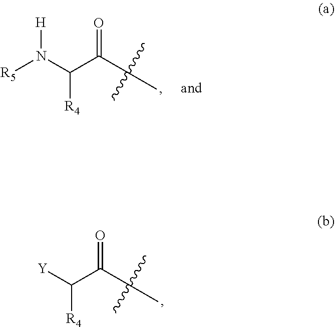
wherein
Y is hydrogen, heterocycle, —NH₂, or C₁-C₄ alkyl;
R₄ is selected from
(i) C₁-C₁₂ alkyl,
(ii) C₁-C₆ heteroalkyl optionally substituted with C₁-C₆ alkyl,
(iii) C₃-C₆ cycloalkyl,
(iv) C₆-C₁₀ aryl,
(v) heterocyclyl,
(vi) C₃-C₁₀ heteroaryl,
(vii) aralkyl, and
(viii) heteroalkylaryl;
R₅ is heteroaryl or —C(═O)—R₁₀, wherein R₁₀ is selected from
(i) C₁-C₁₂ alkyl optionally substituted with C₆-C₁₀ aryl, C₁-C₁₀ heteroaryl, amino, or carboxylic acid,
(ii) C₁-C₁₀ heteroalkyl optionally substituted with C₁-C₆ alkyl or carboxylic acid,
(iii) C₃-C₆ cycloalkyl optionally substituted with C₁-C₆ alkyl, optionally substituted C₆-C₁₀ aryl, optionally substituted C₃-C₁₀ heteroaryl, amino, or carboxylic acid,
(iv) C₆-C₁₀ aryl optionally substituted with C₁-C₆ alkyl, optionally substituted C₆-C₁₀ aryl, optionally substituted C₃-C₁₀ heteroaryl, amino, or carboxylic acid,
(v) heterocyclyl,
(vi) C₃-C₁₀ heteroaryl,
(vii) aralkyl, and
(viii) heteroalkylaryl, and
a pharmaceutically acceptable carrier.

2. The composition according to claim 1, wherein the compound is selected from the group consisting of C1, C2, C3, C4, C5, C6, and stereoisomers, tautomers, or pharmaceutically acceptable salts thereof.

3. The composition according to claim 1, wherein the compound is 4-(((2S,3S)-1-((2-((S)-5-(((2H-tetrazol-5-yl)methyl)carbamoyl)-3-cyclohexyl-2-oxoimidazolidin-1-yl)-2-oxoethyl)amino)-3-methyl-1-oxopentan-2-yl)amino)-4-oxobutanoic acid or a pharmaceutically acceptable salt thereof.

4. The composition according to any one of claims 1-3 formulated for oral administration, nasal administration, topical administration, subcorneal administration, intra-epidermal administration, sub-epidermal administration; or for administration by injection.

5. The composition according to any one of claims 1-4, wherein the topical formulation further comprises a skin penetration enhancer.

6. The composition according to claim 5, wherein the skin penetration enhancer is propylene glycol.

7. The composition according to claim 5, wherein the topical formulation further comprises a viscosity enhancer.

8. The composition according to claim 7, wherein the viscosity enhancer is a crosslinked polyacrylate polymer.

9. The composition according to any one of claims 1-8, wherein the topical formulation has a pH of from about 4 to about 7.4.

10. The composition according to any one of claims 1-8, wherein the topical formulation has a pH of about 6.0.

11. The composition according to any one of claims 1-10, wherein the topical formulation is in the form of a gel comprising from about 0.5 to about 20 mg/mL of a compound of formula (I).

12. The composition according to any one of claims 1-10, wherein the topical formulation is in the form of a gel comprising about 10 mg/mL of a compound of formula (I).

13. A method of treating and/or preventing a blistering and/or peeling of a skin of a subject, comprising administering a therapeutically effective amount of a composition according to any one of claims 1-12 to a subject in need thereof.

14. The method of claim 13, wherein the composition is formulated for oral administration, nasal administration, topical administration, subcorneal administration, intra-epidermal administration, or sub-epidermal administration.

15. The method of claim 13, wherein the composition is formulated for administration by injection.

16. A method of healing a blistered and/or peeled skin of a subject, comprising administering a therapeutically effective amount of a composition according to any one of claims 1-12 to a subject in need thereof.

17. The method according to claim 16, wherein the composition is formulated for oral administration, nasal administration, topical administration, subcorneal administration, intra-epidermal administration, or sub-epidermal administration.

18. The method according to claim 16, wherein the composition is formulated for administration by injection.

19. A method for reducing or preventing blistering and/or peeling of a skin of a subject, comprising administering a therapeutically effective amount of a composition according to any one of claims 1-12 to a subject in need thereof.

20. The method according to claim 19, wherein the composition is formulated for oral administration, nasal administration, topical administration, subcorneal administration, intra-epidermal administration, or sub-epidermal administration.

21. The method according to claim 19, wherein the composition is formulated for administration by injection.
